(19) United States
(12) Patent Application Publication
(10) Pub. No.: US 2021/0008553 A1
(43) Pub. Date: Jan. 14, 2021

(54) METHODS AND SYSTEMS FOR SEPARATING BIOLOGICAL PARTICLES

(71) Applicant: Newomics Inc., Berkeley, CA (US)

(72) Inventors: Daojing WANG, Moraga, CA (US); Yuchao CHEN, Rodeo, CA (US); Pan MAO, Milpitas, CA (US)

(21) Appl. No.: 15/929,373

(22) Filed: Apr. 29, 2020

Related U.S. Application Data
(63) Continuation of application No. PCT/US2018/059879, filed on Nov. 8, 2018.
(60) Provisional application No. 62/583,949, filed on Nov. 9, 2017.

(51) Int. Cl. B01L 3/00; G01N 33/49

(52) U.S. Cl. CPC B01L 3/502753; G01N 33/491; B01L 2300/0864; B01L 2200/12; B01L 2200/0652

(57) ABSTRACT

The present disclosure provides methods and systems for separating one or more target analytes from a fluid sample. The systems may comprise a microfluidic device. The microfluidic device may comprise a fluidic channel having an array of obstacles disposed therein. The array of obstacles may be oriented at an angle greater than 0° relative to a direction of a fluid flow in the fluidic channel. The array of obstacles may be configured to separate the target analytes from the fluid upon flow of the fluid through the fluidic channel. The methods of the present disclosure may comprise separating target analytes from a fluid using a microfluidic device comprising obstacles disposed in a fluidic channel of the device. The target analytes may be separated with a high efficiency, sensitivity and/or specificity.

### CROSS-REFERENCE

This application is a continuation of International Patent Application No. PCT/US2018/059879, filed Nov. 8, 2018, which claims the benefit of U.S. Provisional Patent Application No. 62/583,949, filed Nov. 9, 2017, each of which is incorporated herein by reference in its entirety.

### STATEMENT AS TO FEDERALLY SPONSORED RESEARCH

This invention was made with government support under grant numbers ES022360, ES022360, GM109682, AT008297, AG046025, and AI106100 and contract number HHSN261201300033C awarded by the National Institutes of Health. The government has certain rights in the invention.

### BACKGROUND

Separation and sorting of biological particles may be important for a variety of biomedical applications, including diagnostics, therapeutics, or fundamental cell biology. For example, understanding the causes underlying diseases may require separation of specific biological molecules or particles from complex samples, such as biofluids. Microfluidic-based methods and systems may be used for separating, capturing, detecting or analyzing biological molecules or particles.

### SUMMARY

Separation and sorting of biological particles may be important for a variety of biomedical applications, including diagnostics, therapeutics, or fundamental cell biology. Biological particles may include particles of biological origin. Non-limiting examples of biological particles may include cells or components thereof (e.g., nuclei), viruses, bacteria, proteins, carbohydrates, nucleic acid molecules (such as deoxyribonucleic acid (DNA), ribonucleic acid (RNA)), lipid, or combinations thereof. In some cases, cells may be senescent cells.

Cellular senescence is a state of permanent cell cycle arrest due to genotoxic stresses and may be involved in organismal aging and tumorigenesis. Thus, senescent cell may be an important biomarker for aging as well as genotoxic stresses such as ionizing radiation. However, the small number of senescent cells in biofluids such as whole blood may limit their quick and sensitive detection.

Cellular senescence may play an important role in organismal aging and age-related diseases. However, it may be challenging to isolate low numbers of senescent cells from small volumes of biofluids for downstream analysis.

Recent animal studies have shown the potential of therapeutic targeting of senescent cells for anti-aging and age-related diseases. Because pathways up- or down-regulated in senescent cells, such as those involving p16, p21, and p53, may also function at various degrees in their healthy counterparts throughout the tissues and organs, conventional methods that target these pathways with small molecules and protein drugs may result in side effects in humans.

Different microfluidic techniques may be utilized for cell separation based on their physical properties (size, deformability, density, etc.), including filtration, deterministic lateral displacement (DLD), inertial flow, and acoustofluidics. Filtration may be used to process undiluted whole blood for rare cell separation and easily scaled up for a higher throughput. However, several challenges may exist. In dead-end flow filtration which may have the flow direction perpendicular to the filter surface, common issues may comprise the clogging and saturation of the filter, resulting in a lower separation efficiency, sample purity, and device robustness. A periodic reversed flow or fluidic oscillation may be adopted to address clogging. However, it may reduce the separation throughput and operation simplicity. Additionally, cell integrity may be decreased as they squeeze through the filtration pores, which may result in the changes of cell cytoskeleton. Crossflow filtration in microfluidics with a flow direction parallel to the filter surface may be used to decrease cell damage and clogging issue, and a shear force may be generated to bring the bigger particles to the downstream instead of entering the filtration pores. However, to ensure effective cell separation in a parallel-flow configuration, the crossflow filtration may require a much longer channel with a throughput lower than 1 milliliter/hour (mL/h). Despite the low throughput for microfluidic devices, a higher throughput (e.g. >1 mL/min) may be highly desired in order to process a large volume of complex samples such as whole blood samples. High throughput may be particularly challenging for a continuous flow due to difficulties in system integration and fluidic control for multiplexing on a microfluidic chip. Accordingly, recognized herein is a need for a platform that can achieve specificity, sensitivity, and throughput for isolation and removal of diverse “toxic” cells from biofluids.

The present disclosure provides effective isolation approaches for senescent-cell-based point-of-care diagnostics, such as radiation biodosimetry. The present disclosure provides methods and systems that can selectively remove senescent cells in a high-throughput manner.

The present disclosure provides methods and systems for separating, capturing, detecting and/or analyzing biological particles, molecules or components. The methods and systems may be used for radiation biodosimetry, radiological/nuclear medical countermeasures, anti-aging and anti-cancer therapies. The methods and systems may comprise the use of microfluidic-based platform. The microfluidic-based platform may comprise microfluidic devices. The microfluidic devices may comprise one or more microfluidic channels which may comprise one or more obstacles. The one or more obstacles may comprise an array of obstacles. The array of obstacles may be a three-dimensional (3D) array. The microfluidic devices may comprise an integrated microfluidic chip. The microfluidic devices may be monolithic. The microfluidic chip may be used for on-chip and online biological particle separation. The microfluidic devices may be used for single-cell analysis. The microfluidic devices may be used for biological particle separation in small volumes of fluid samples. In some cases, the microfluidic devices may be used for ultrahigh-throughput size-based isolation and removal of diverse cells in biofluids. The microfluidic devices of the present disclosure can be used for processing samples of various volumes and/or quantities. For example, the microfluidic devices can be used for processing both small-volume and large-volume samples.

Additionally or alternatively, the microfluidic-based platform may comprise multi-unit, large-dimension microfluidic chips. Such microfluidic chips may be used for ultrahigh-throughput parallel particle isolation and removal. The samples from which the particles are removed may have a large volume.

An aspect of the present disclosure provides a microfluidic device comprising: a fluidic channel; and an array of obstacles disposed in the fluidic channel, wherein the array of obstacles is oriented at an angle greater than 0° relative to a direction of a fluid flow in the fluidic channel; wherein the array of obstacles is configured to separate one or more target analytes from a fluid flowing through the fluidic channel.

In some embodiments, the angle is between about 1° and 85°. In some embodiments, the angle is between about 5° and 30°. In some embodiments, a distance between the array of obstacles and a side wall of the fluidic channel increases along the direction of the fluid flow. In some embodiments, at least one obstacle of the array of obstacles is adjacent to a side wall of the fluidic channel. In some embodiments, individual obstacles of the array of obstacles have a quadrilateral cross-section. In some embodiments, the quadrilateral cross-section is a parallelogram cross-section. In some embodiments, the array of obstacles is slanted in vertical or various angular directions. In some embodiments, an average spacing size between obstacles of the array is between about 100 nanometers and 100 micrometers (μm). In some embodiments, the average spacing size is between about 1 μm and 100 μm. In some embodiments, individual obstacles of the array have spaces configured to separate the one or more target analytes from the fluid. In some embodiments, the array of obstacles has a height less than or equal to a height of the fluidic channel. In some embodiments, the array of obstacles is configured to direct the one or more target analytes to flow at a direction different from the direction of the fluid flow. In some embodiments, the array of obstacles is configured to separate the one or more target analytes from the fluid based at least partially on a size of the one or more target analytes. In some embodiments, the size of the one or more target analytes is greater than or equal to a threshold value. In some embodiments, the one or more target analytes comprise biological particles. In some embodiments, the one or more target analytes comprise cells. In some embodiments, the cells comprise senescent cells. In some embodiments, the fluid comprises a biofluid. In some embodiments, the biofluid comprises whole blood. In some embodiments, the whole blood is undiluted or diluted. In some embodiments, the array of obstacles comprises microstructures. In some embodiments, the microstructures comprise micropillars. In some embodiments, the microstructures are three-dimensional (3D) microstructures. In some embodiments, obstacles of the array of obstacles have an average size between about 1 μm and about 100 μm. In some embodiments, at least a subset of the array of obstacles deforms when a flow rate of the fluid is greater than a threshold value. In some embodiments, obstacles of the array of obstacles are non-porous. In some embodiments, the microfluidic device further comprises one or more fluid inlets in fluidic communication with the fluidic channel. In some embodiments, the microfluidic device further comprises at least a first fluid outlet and a second fluid outlet in fluidic communication with the fluidic channel. In some embodiments, the first fluid outlet is configured to receive the one or more target analytes and the second fluid outlet is configured to receive the fluid absent the one or more target analytes. In some embodiments, the microfluidic device further comprises a fluidic component in fluidic communication with the fluidic channel. In some embodiments, the fluidic component is configured to receive and remove the one or more target analytes from the fluidic channel. In some embodiments, the fluidic component is a tubing. In some embodiments, the microfluidic device further comprises an additional fluidic channel in fluidic communication with the fluidic channel. In some embodiments, the additional fluidic channel is configured to receive and retain the one or more target analytes. In some embodiments, the microfluidic device further comprises an additional array of obstacles disposed therein. In some embodiments, the additional array of obstacles is configured to capture the one or more target analytes. In some embodiments, each of the additional array of obstacles has an opening. In some embodiments, the opening has a dimension greater than or equal to a size of the one or more target analytes. In some embodiments, the microfluidic device comprises a plurality of microfluidic channels each comprising a different array of obstacles. In some embodiments, each of the plurality of microfluidic channels is configured to separate a given type of target analytes from a fluid flowing therethrough. In some embodiments, the microfluidic device further comprises a body structure comprising a substrate. In some embodiments, the fluidic channel is disposed within the substrate. In some embodiments, the microfluidic device further comprises one or more fluidic pumps configured to transport fluids within the microfluidic device. In some embodiments, the one or more fluidic pumps comprise a plurality of valves.

Another aspect of the present disclosure provides a microfluidic device comprising: a fluidic channel; and an array of obstacles disposed in the fluidic channel; wherein the array of obstacles is configured to separate one or more particles from a fluid flowing through the fluidic channel with an efficiency of greater than about 70% at a flow rate of greater than or equal to about 250 milliliters/hour (mL/hr).

In some embodiments, the fluid is whole blood. In some embodiments, the one or more particles comprise senescent cells. In some embodiments, the one or more particles have an average size greater than or equal to about 25 micrometers. In some embodiments, the fluid has a volume more than or equal to about 30 milliliters. In some embodiments, the efficiency is greater than about 85%. In some embodiments, the flow rate is greater than or equal to about 300 ml/hr.

Another aspect of the present disclosure provides a microfluidic device comprising: a fluidic channel; and an array of obstacles disposed in the fluidic channel; wherein the array of obstacles is configured to separate one or more senescent cells from a fluid having a volume less than or equal to about 1 milliliter (mL) at an efficiency greater than about 70% upon flow of the fluid through the fluidic channel.

In some embodiments, the efficiency is greater than about 85%. In some embodiments, less than 25 mol % of non-senescent cells are separated from the fluid. In some embodiments, the fluid comprises biofluids. In some embodiments, the biofluids comprise whole blood or bone marrow. In some embodiments, the whole blood is undiluted. In some embodiments, the volume is less than or equal to about 50 microliters (μL). In some embodiments, the volume is less than or equal to about 25 μL. In some embodiments, the volume is less than or equal to about 5 μL. In some embodiments, the array of obstacles comprises microstructures. In some embodiments, the microstructures comprise micropillars. In some embodiments, the microstructures are three-dimensional (3D) microstructures. In some embodiments, obstacles of the array of obstacles are nonporous. In some embodiments, an average spacing size between obstacles of the array is between about 100 nanometers and 100 micrometers (μm). In some embodiments, the average spacing size is between about 1 μm and 100 μm. In some embodiments, the one or more senescent cells comprise senescent T cells, different kinds of white blood cells, microphages, lung, breast, colon, prostate, gastric, hepatic, ovarian, esophageal, or bronchial epithelial or stromal cells, senescent skin epithelial or stromal cells, senescent glial cells, senescent vascular endothelial or stromal cells, or combinations thereof.

Another aspect of the present disclosure provides a method comprising: (a) directing a fluid comprising one or more target analytes into a microfluidic device, the microfluidic device comprising: a fluidic channel; and an array of obstacles disposed in the fluidic channel, wherein the array of obstacles is oriented at an angle greater than 0° relative to a direction of a fluid flow in the fluidic channel; (b) directing the fluid to flow through the fluidic channel; and (c) separating at least a portion of the one or more target analytes from the fluid using the array of obstacles upon flow of the fluid through the fluidic channel.

In some embodiments, the angle is between about 1° and 85°. In some embodiments, the angle is between about 5° and 30°. In some embodiments, a distance between the array of obstacles and a side wall of the fluidic channel increases along the direction of the fluid flow. In some embodiments, at least one obstacle of the array of obstacles is adjacent to a side wall of the fluidic channel. In some embodiments, individual obstacles of the array of obstacles have a quadrilateral cross-section. In some embodiments, the quadrilateral cross-section is a parallelogram cross-section. In some embodiments, the one or more target analytes comprise biological particles. In some embodiments, the one or more target analytes comprise cells. In some embodiments, the cells comprise senescent cells. In some embodiments, the method further comprises directing an additional fluid into the microfluidic device. In some embodiments, the additional fluid is a sheath fluid. In some embodiments, the sheath fluid comprises a buffer. In some embodiments, the method further comprises capturing the at least the portion of the one or more target analytes. In some embodiments, the method further comprises subjecting the at least the portion of the one or more target analytes to further analyses. In some embodiments, the method further comprises removing the at least the portion of the one or more target analytes from the microfluidic device. In some embodiments, the at least the portion of the one or more target analytes is separated at a sensitivity of at least about 70%. In some embodiments, the at least the portion of the one or more target analytes is separated at a specificity of at least about 70%. In some embodiments, the method further comprises detecting the at least the portion of the one or more target analytes.

Another aspect of the present disclosure provides a method comprising: (a) directing a fluid comprising one or more particles into a microfluidic device, the microfluidic device comprising: a fluidic channel; and an array of obstacles disposed in the fluidic channel; (b) directing the fluid to flow through the fluidic channel; and (c) upon flow of the fluid through the fluidic channel, using the array of obstacles to separate the one or more particles from the fluid with an efficiency of greater than about 70% at a flow rate of greater than or equal to about 250 milliliters/hour (ml/hr).

In some embodiments, the fluid is whole blood. In some embodiments, the one or more particles comprise senescent cells. In some embodiments, the one or more particles have an average size greater than or equal to about 25 micrometers. In some embodiments, the fluid has a volume greater than or equal to about 30 milliliters. In some embodiments, the efficiency is greater than about 85%. In some embodiments, the flow rate is greater than or equal to about 300 ml/hr.

Another aspect of the present disclosure provides a method comprising: (a) directing a fluid having a volume less than or equal to about 1 milliliter (mL) into a microfluidic device, wherein the fluid comprise one or more senescent cells and wherein the microfluidic device comprises: a fluidic channel; and an array of obstacles disposed in the fluidic channel; (b) directing the fluid to flow through the fluidic channel; and (c) using the array of obstacles to separate at least a portion of the one or more senescent cells from the fluid at an efficiency greater than about 70% upon flow of the fluid through the fluidic channel.

In some embodiments, the efficiency is greater than about 85%. In some embodiments, less than 25 mol % of non-senescent cells are separated from the fluid. In some embodiments, the fluid comprises biofluids. In some embodiments, the biofluids comprise whole blood or bone marrow. In some embodiments, the whole blood is undiluted. In some embodiments, the volume is less than or equal to about 50 microliters (μL). In some embodiments, the volume is less than or equal to about 25 μL. In some embodiments, the volume is less than or equal to about 5 μL. In some embodiments, the method further comprises directing an additional fluid into the microfluidic device. In some embodiments, the additional fluid is a sheath fluid. In some embodiments, (c) is conducted based at least partially on a size of the one or more senescent cells. In some embodiments, the array of obstacles comprises microstructures. In some embodiments, the microstructures comprise micropillars. In some embodiments, the microstructures are three-dimensional (3D) microstructures. In some embodiments, obstacles of the array of obstacles are nonporous. In some embodiments, an average spacing size between obstacles of the array is between about 100 nanometers and 100 micrometers (μm). In some embodiments, the average spacing size is between about 1 μm and 100 μm. In some embodiments, (c) comprises using the array of obstacles to cause the at least the portion of the one or more senescent cells to flow at a direction that is different from a direction of the fluid in the fluidic channel. In some embodiments, the method further comprises capturing the at least the portion of the one or more senescent cells. In some embodiments, the method further comprises subjecting the at least the portion of the one or more senescent cells to further analyses. In some embodiments, the method further comprises removing the at least the portion of the one or more senescent cells from the microfluidic device. In some embodiments, the at least the portion of the one or more senescent cells is separated at a sensitivity of at least about 70%. In some embodiments, the at least the portion of the one or more senescent cells is separated at a specificity of at least about 70%. In some embodiments, the method further comprises detecting the at least the portion of the one or more senescent cells. In some embodiments, the detecting is at a single-cell resolution. In some embodiments, the one or more senescent cells comprise senescent T cells, different kinds of white blood cells, microphages, lung, breast, colon, prostate, gastric, hepatic, ovarian, esophageal, or bronchial epithelial or stromal cells, senescent skin epithelial or stromal cells, senescent glial cells, senescent vascular endothelial or stromal cells, or combinations thereof.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIGS. 1A-1C illustrate sample chips and uses thereof (e.g., for processing of senescent cells). FIG. 1A shows two types of sample chips: (i) a chip with a 3D filter array and a cell trap array for capture and single cell analysis of senescent cells in blood; and (ii) a chip with a 3D filter array connected with a tubing at the outlet for removal of senescent cells from blood. Zoom-in regions show schematic separation and trapping of red blood cell (RBC), while blood cell (WBC), and senescent cells, along with two types of pillar shapes A and B. FIG. 1B shows mechanism of size-based cell separation with a 3D filter array: (i) the top view of the filters with force analysis on the x and y directions; (ii) the side view of the filters on the x and z directions; and (iii) the perspective view of the filters on the x, y, and z directions. FIG. 1C shows images of the experimental setup and operation: (i) an actual-size image of a chip relative to a US dime; (ii) the experimental setup showing tubing connections and pumps; and (iii) a chip in operation of processing whole blood samples. Scale bar represents 5 mm in (iii);

FIGS. 2A-2C illustrate operation of sample chips. FIG. 2A shows time-lapse images of (i) a bead and (ii) a cell, roll down on a 3D filter array. FIG. 2B shows (i) images of undiluted whole blood passing through a 3D-filter array without clogging (ii) stacking images showing complete separation of 18 μm beads from 10 μm beads (left), and separation of MSCs from undiluted whole blood (right). FIG. 2C shows images of cell trap array located at (i) blood outlet and (ii) senescent cell outlet, after separation of MSCs from whole blood. Cells with blue color are senescent cells (SA-β-gal positive); (iii) phase-contrast and fluorescence imaging of CD45 labeling for identification of senescent MSCs and WBCs. Scale bars represent 50 μm in FIG. 2A and FIG. 2C, 150 μm in (FIG. 2B-i), and 100 μm in (FIG. 2B-ii), respectively. FIGS. 2A-2C are shown as the corresponding zoom-in regions on a sample schematic chip for presentation clarity;

FIGS. 3A-3D show validation of sample chips for size-based separation. FIG. 3A shows schematic of a sample chip for characterization with beads or cells. FIG. 3B shows recovery of beads from outlet (iii), for four sizes of beads (6 μm, 10 μm, 15 μm, and 18 μm) mixed to characterize three types of chips (z-direction only filter, 4 μm 3D filter, and 13 μm 3D filter) at three flow rates (1 mL/h, 3 mL/h, and 5 mL/h). FIG. 3C shows recovery of WBCs isolated from whole blood from outlet (iii), with three types of chips at three flow rates as in FIG. 3B. FIG. 3D shows recovery of basal MSCs from undiluted whole blood at outlet (iv), with three types of chips at a flow rate of 3 mL/h;

FIGS. 4A-4C show application of sample chips for analysis of senescent cells in biofluids. FIG. 4A shows senescence-associated beta-galactosidase (SA-β-gal) staining of MSCs cultured on a 12-well plate. The MSCs are treated with different doses of hydrogen peroxide (H₂O₂, 0, 100, 200 μM) and X-ray (0, 1, 4 Gy), and analyzed 3 days and 6 days after the treatments. Cells stained blue are SA-β-gal positive. FIG. 4B shows quantitation of SA-β-gal staining of MSCs on culture dish (i) and MSCs isolated from human whole blood on the chip (ii). The percentage of SA-β-gal positive is calculated for the stained blue-stained MSCs among the total MSCs. FIG. 4C shows isolation and analysis of senescent cells from mouse bone marrow after total body irradiation (TBI) of 0, 1 Gy, 4 Gy, and 6 Gy X-ray radiation (n=3), respectively. Scale bar represents 100 μm in FIG. 4A;

FIGS. 5A-5D show application of sample chips for removal of senescent cells from whole blood. FIG. 5A shows schematic of removal of senescent cells from whole blood, using a 13 μm 3D filter. FIG. 5B shows cell size distribution of basal MSCs and senescent MSCs, 3 and 6 days after treatment with different doses of hydrogen peroxide and X-ray. FIG. 5C illustrates enrichment of senescent cells at outlet (iv) using a 13 μm 3D filter chip. In comparison, original MSCs without separation, MSCs directly captured on a cell trap array without a 3D filter, and MSCs processed on a chip with a 4 μm 3D filter are also studied. FIG. 5D shows removal of senescent cells from undiluted whole blood using a 13 μm 3D filter chip via outlet (iv);

FIGS. 6A-6E show sample ultrahigh-throughput chips for removal of senescent cells from human whole blood. FIG. 6A shows schematic of the high-throughput chip. Five large-dimension channels are stacked and integrated for parallel processing. FIG. 6B shows image of a sample high-throughput chip compared to a regular-size single-unit device. FIG. 6C shows cross-section view of the multi-layer and multi-channel chip showing integration of five channels in five vertical layers. FIG. 6D shows microscope images showing MSCs before separation, spiked in blood, and after separation. FIG. 6E shows quantification of the numbers of basal MSCs and senescent MSCs spiked into whole blood, before and after removal of senescent MSCs using our chip. Scale bars represent 10 mm in FIG. 6B and 50 μm in FIG. 6D;

FIGS. 7A-7D show flow simulation inside a microfluidic channel. FIG. 7A shows simulation of flow velocity in a 2D filter array. FIG. 7B shows simulation of flow velocity in a 3D filter array. FIG. 7C shows plot of flow velocity along the dash line in FIG. 7A. FIG. 7D shows plot of flow velocity along the dash line in FIG. 7B; and

FIG. 8 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “obstacle,” as used herein, generally refers to any structure that is capable of obstructing a flow of a fluid, impeding the flow of the fluid, and/or diverting the flow of the fluid. In some examples, the obstacle is pillar. The pillar may have dimensions on the order of nanometers (i.e., nanopillar) or micrometers (i.e., micropillar). The obstacle may be distributed in an array of a plurality of obstacles (or array of obstacles). The obstacle may have various shapes. The obstacle may have a cross-section that is circular, triangular, quadrilateral, pentagonal, hexagonal, or any combination of shapes or partial-shapes thereof.

An array of obstacles may comprise a plurality of obstacles that have regular or substantially regular shapes and/or sizes. In some examples, the plurality of obstacles have a coefficient of variation of less than or equal to about 30%, 25%, 20%, 15%, 10%, 5%, 4%, 3%, 2%, 1%, or less. As an alternative, the plurality of obstacles may have irregular or substantially irregular shapes and/or sizes.

In some examples, the plurality of obstacles is generally distributed in an array that is angled with respect to the general direction of flow into the array. Such array may not include other obstacles. For example, the plurality of obstacles are oriented at an angle greater than about 0°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85° or more with respect to the general direction of flow into the array (e.g., a vector parallel to one or more axes directed through one or more subsets of the plurality of particles may be oriented at an angle greater than 0° with respect to a vector oriented along the general direction of flow). Such angle may be constant with respect to the general direction of flow. Alternatively, such angle may vary along the general direction of flow (e.g., the angle may increase or decrease along the general direction of flow).

Flow directed in an array of obstacles may be laminar. As an alternative, the flow may be turbulent.

Whenever the term “at least,” “greater than,” or “greater than or equal to” precedes the first numerical value in a series of two or more numerical values, the term “at least” or “greater than” applies to each one of the numerical values in that series of numerical values.

Whenever the term “no more than,” “less than,” or “less than or equal to” precedes the first numerical value in a series of two or more numerical values, the term “no more than” or “less than” applies to each one of the numerical values in that series of numerical values.

Provided herein are methods and systems for separating, isolating, capturing, detecting and/or analyzing target analytes. The target analytes may comprise biological particles. Biological particles may include any particles of biological origin. Non-limiting examples of biological particles may include cells or components thereof, viruses, bacteria, proteins, carbohydrates, nucleic acid molecules (such as deoxyribonucleic acid (DNA), ribonucleic acid (RNA)), lipid, or combinations thereof. Non-limiting examples of cells may include, tumor cells, red blood cells, white blood cells (such as T cells, B cells, and helper T cells), infected cells, trophoblasts, fibroblasts, stem cells, epithelial cells, infectious organisms (e.g., bacteria, protozoa, and fungi), cancer cells, bone marrow cells, fetal cells, progenitor cells, foam cells, mesenchymal cells, immune system cells, endothelial cells, endometrial cells, connective tissue cells, trophoblasts, bacteria, fungi, or pathogens, or combinations thereof. In some cases, cells may comprise senescent cells. Senescent cells may comprise senescent cells of any type of above-mentioned cells. For example, senescent cells may comprise senescent T cells, senescent white blood cells, senescent microphages, senescent lung, breast, colon, prostate, gastric, hepatic, ovarian, esophageal, or bronchial epithelial or stromal cells, senescent skin epithelial or stromal cells, senescent glial cells, senescent vascular endothelial or stromal cells, or combinations thereof.

### Systems

Systems of the present disclosure may comprise microfluidic devices. A microfluidic device, as provided herein, may comprise a body structure. The body structure may be a single layer or multi-layer structure. The body structure may comprise a substrate. The substrate may comprise a fluidic channel disposed therein. The fluidic channel may have an aspect ratio (a ratio of channel length to an average cross-sectional dimension of the channel) that is greater than or equal to about 2, 3, 4, 5, 6, 7, 8, 9, 10, 11, 12, 13, 14, 15, 16, 17, 18, 19, 20, 22, 24, 26, 28, 30, 32, 34, 36, 38, 40, 45, 50, 55, 60, 65, 70, 75, 80, 85, 90, 95, 100, or more. In some cases, the aspect ratio may be less than or equal to about 200, 180, 160, 140, 120, 100, 80, 70, 60, 50, 40, 30, 20 or less. In some cases, the aspect ratio may be between any of the two values described above and elsewhere herein, for example, between about 15 and 30.

The fluidic channel may comprise one or more obstacles disposed therein. The one or more obstacles may be a plurality of obstacles. The obstacles may be any structures that may have an impact or effect on a fluid or components thereof, while the fluid flows through the microfluidic channel. For example, the obstacles may delay, alter or impede a fluid flow (e.g., flow rate of the fluid flow) in the channel. The obstacles may comprise obstacles associated with or immobilized on a surface (e.g., bottom, top or side walls) of the microfluidic channel. The surface may be a substrate or a side wall of the fluidic channel. The obstacles may be extended partially or fully across the channel. The obstacles may be extended partially or fully along a height of the fluidic channel. The obstacles may have an average height that is less than or equal to an average height (or depth) of the microfluidic channel. The obstacles may have an average height that is greater than or equal to about 1 micrometer (micron, μm), 2 μm, 5 μm, 10 μm, 12 μm, 14 μm, 16 μm, 18 μm, 20 μm, 22 μm, 24 μm, 26 μm, 28 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, or more. The obstacles may have an average height that is less than or equal to about 150 μm, 125 μm, 100 μm, 90 μm, 80 μm, 70 μm, 60 μm, 50 μm, 45 μm, 40 μm, 35 μm, 30 μm, 25 μm, 20 μm, 15 μm, 10 μm, 8 μm, 6 μm, 4 μm, 1 μm, or less. In some cases, the obstacles have an average size that falls between any of the two values described above or elsewhere herein, for example, between about 30 μm and 35 μm.

The obstacles may be microstructures, nanostructures or combinations thereof. The obstacles may be three-dimensional (3D) structures. The 3D obstacles may be obstacles that have openings in x-, y-, and z-directions. The 3D obstacles may deform in x-, y-, and/or z-directions upon application of a pressure. The pressure may be resulted from a fluid flow. The pressure may change with flow rate of the fluid flow. The obstacles may comprise micropillars. The micropillars may be 3D micropillars. The obstacles may have an average size that is greater than or equal to about 1 μm, 2 μm, 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, or more. The obstacles may have an average size that is less than or equal to about 200 μm, 180 μm, 160 μm, 140 μm, 120 μm, 100 μm, 90 μm, 80 μm, 60 μm, 50 μm, 40 μm, 30 μm, 20 μm, 15 μm, 10 μm, 8 μm, 6 μm, 4 μm, 2 μm, 1 μm, or less. In some cases, the obstacles may have an average size that is between any of the two values described above and elsewhere herein, for example, between about 1 μm and 100 μm.

The obstacles may be porous or nonporous. The obstacles may be solid, or semi-solid. Materials suitable for forming the obstacles may include polymers, metals, ceramics, carbons, or combinations thereof.

The dimensions and geometry of the obstacles may vary. The obstacles may have regular, or irregular cross sections. In some cases, the obstacles comprise one or more subsets of the obstacles. The one or more subsets of the obstacles may comprise obstacles having cross sections that are the same as or different from one another. In some cases, the obstacles have quadrilateral cross sections such as parallelogram cross sections.

In some cases, at least a subset of the obstacles may be slanted. The subset of the obstacles may be slanted in vertical direction. The subset of the obstacles may be slanted in vertical direction that is perpendicular to a plane of a substrate within which a microfluidic channel is disposed. The subset of the obstacles may be slanted in various angular directions. The various angular directions may be any directions that are angled with respect to, e.g., a plane of a substrate within which a microfluidic channel is disposed. The angle may be between about 0° and 90°.

The one or more obstacles may comprise an array of obstacles. The array of obstacles may comprise any number of obstacles (e.g., greater than or equal to about 5, 10, 15, 20, 25, 30, 35, 40, 45, 50, 55, 60, 65, 70, 75, 80, 85, 90, 95, 100 or more obstacles). The array of obstacles may be angled relative to a direction of a fluid flow in the microfluidic channel. The array of obstacles may be aligned or oriented to a direction that is angled relatives to the direction of the fluid flow. There may be an angle between the direction along which the array of obstacles is aligned and the direction of the fluid flow. The angle may be an oblique angle. The angle may be between about 0° and about 90°. In some cases, the angle may be greater than about 0°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85° or more. In some cases, the angle may less than about 90°, 85°, 80°, 75°, 70°, 65°, 60°, 55°, 50°, 45°, 40°, 35°, 30°, 25°, 20°, 15°, 10°, 9°, 7°, 5°, 3°, 1°, or less. In some cases, the angle may be between any of the values described above or elsewhere herein, for example, between about 20° and 30°. In some cases, all of the obstacles are angled relative to the direction of the fluid flow.

The obstacles may be spaced from one another. An average spacing size of the obstacles (e.g., an average space between adjacent obstacles) may vary. The average spacing size may be adjusted depending upon a variety of factors, including such as dimension of the microfluidic channel, number of obstacles disposed in the microfluidic channel, sample volume, sizes, dimensions, geometries of target analytes, fluid flow rate, or combinations thereof. In some cases, the obstacles may have an average spacing size greater than or equal to about 10 nanometers (nm), 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1 μm, 5 μm, 10 μm, 20 μm, 30 μm, 40 μm, 50 μm, 60 μm, 70 μm, 80 μm, 90 μm, 100 μm, or more. In some cases, the average spacing size may be less than or equal to about 200 μm, 180 μm, 160 μm, 140 μm, 120 μm, 100 μm, 85 μm, 75 μm, 65 μm, 55 μm, 45 μm, 35 μm, 25 μm, 15 μm, 5 μm, 1 μm, 850 nm, 750 nm, 650 nm, 550 nm, 450 nm, 350 nm, 250 nm, 150 nm, 100 nm, or less. In some cases, the average spacing size may be any of the values described above or elsewhere herein, for example, between about 100 nm and 100 μm.

The one or more obstacles may comprise a plurality of arrays of obstacles. In some cases, the microfluidic channel may have a uniform cross sectional dimension, and the plurality of the obstacles arrays may be disposed within the microfluidic channel. In some cases, the microfluidic channel may comprise one or more sections along a length of the channel. The one or more sections may have the same or different cross sectional dimensions. At least one obstacle array may be disposed within each section of the microfluidic channels. The obstacle array disposed in different sections of the microfluidic channel may be the same or may be different. The obstacle array disposed in different sections of the microfluidic channel may comprise obstacles that are of the same or different sizes, shapes, geometries, and/or cross-sections. For example, a microfluidic channel may comprise at least two sections each comprising an array of obstacles. The obstacle arrays disposed in different sections may comprise different number of obstacles. Each array may have a distinctive size and geometry (i.e., obstacles comprised in one array may have a size and/or cross section different from those comprised in the other array). The two sections may each be configured to separate or isolate a specific type of target analyte comprised in a fluid while the fluid is flowing through the microfluidic channel.

In some cases, the microfluidic device comprises a plurality of fluidic channels. For at least a subset of the plurality of the fluidic channels, each individual fluidic channel may comprise a different array of obstacles disposed therein. The different obstacles arrays may differ from one another in number of obstacles, size of the obstacles, cross sections of the obstacles, dimension of the obstacles, configuration of the array, and/or direction along which the array is oriented. The plurality of the fluidic channels may be configured to separate different target analytes from a given sample. The plurality of the fluidic channels may be configured to separate a given target analyte from different fluid samples. The plurality of the fluidic channels may be configured to process a plurality of fluid samples simultaneously. The plurality of the fluidic channels may be configured to simultaneously or substantially simultaneously process at least about 2, 3, 4, 5, 6, 7, 8, 9, 10, 12, 14, 16, 18, 20, 25, 30, 35, 40, 45, 50, 60, 70, 80, 90, 100 or more samples.

There may exist a distance between the array of obstacles and a side wall of the microfluidic channel. In some cases, at least one obstacle disposed in the microfluidic channel is adjacent to a side wall of the microfluidic channel. For example, a distance between at least one obstacle disposed in the microfluidic channel and a side wall of the channel may be less than or equal to about 1 μm, 0.5 μm, 0.4 μm, 0.3 μm, 0.2 μm, 0.1 μm, 0.05 μm, or less. The distance between the array of obstacles and a side wall of the microfluidic channel may increase along a direction of fluid flow.

The obstacles (e.g., the array of obstacles) may be configured to separate or isolate one or more target analytes from a fluid flowing through the microfluidic channel. The target analytes may comprise biological particles. The biological particles may be any biological particles described above or elsewhere herein. The target analytes may comprise cells, including any types of cells described above or elsewhere herein. In some cases, the cells comprise senescent cells. The fluid comprising the target analytes may comprise biofluids. The biofluids may be any types of biofluids which may be obtained from a subject. A subject may be any living being comprised of at least one cell. A subject can be a single cell organism or a multi-cellular organism, such as a mammal, a non-mammal (e.g., a bird), or a plant (e.g., a tree). A subject may be a mammal, such as, for example, a human or an animal such as a primate (e.g., a monkey, chimpanzee, etc.), a domesticated animal (e.g., a dog, cat, etc.), farm animal (e.g., goat, sheep, pig, cattle, horse, etc.), or laboratory animal (e.g., mouse, rat, etc.). A subject may be a patient. A subject may be an individual that has or is suspected of having a disease. Examples of subjects may include, but not limited to, humans, mammals, non-human mammals, rodents, amphibians, reptiles, canines, felines, bovines, equines, goats, ovines, hens, avines, mice, rabbits, insects, slugs, microbes, bacteria, parasites, or fish. In some cases, the subject may be a patient who is having, suspected of having, or at a risk of developing a disease or disorder, or encountering an environmental contamination. The biofluids may comprise naturally occurring fluids (e.g., blood, sweat, tears, ear flow, sputum, lymph, bone marrow suspension, urine, saliva, semen, vaginal flow, cerebrospinal fluid, cervical lavage, brain fluid, ascites, milk, secretions of the respiratory, intestinal or genitourinary tract, amniotic fluid, and water samples), fluids into which cells have been introduced (e.g., culture media and liquefied tissue samples), or combinations thereof. In some cases, the biofluids comprise whole blood. The whole blood may be diluted or undiluted.

The obstacles may be configured to separate or isolate the target analytes using the spaces between the obstacles. The separation or isolation of the target analytes may be based at least partially on sizes or dimensions of the target analytes. It may be desirable that the target analytes have a size or dimension that is greater than or equal to a pre-determined threshold value. The pre-determined threshold value may be identified using reference particles. The reference particles may be directed to flow through a microfluidic channel having obstacles disposed therein. The obstacles may have a known spacing size. Upon flow of the reference particles through the microfluidic channel, a threshold value may be identified. As the separation occurs, the obstacles may be configured to direct the target analytes to flow along a direction that is different from the direction of the fluid flow.

In some cases, at least a subset of the obstacles may have certain flexibility. The obstacles may function as cantilevers, which only have one end fixed. For example, the obstacles may be immobilized on a surface (e.g., channel bottom surface or top surface) of the microfluidic channel and may have a height that is less than or equal to a height (or depth) of the microfluidic channel. Such flexibility of the obstacles may allow for deformation of the obstacles under certain situations, for example, when a flow rate of the fluid comprising the target analytes is greater than a threshold value. In some cases, at least a subset of the obstacles may deform when experiencing a fluidic pressure, which may create shutters in the vertical direction responsive to the fluidic pressure. The shutters may help to release backpressure, thus reducing clogging in the microfluidic channel.

As provided herein, the microfluidic devices may comprise one or more additional components. For example, the microfluidic devices may comprise one or more fluid inlets. The fluid inlets may be in fluidic communication with the fluidic channel. The fluid inlets may be configured to receive fluids and direct the fluids into the microfluidic channel. The fluid inlets may comprise at least a first fluid channel and a second fluid channel. The first fluid channel and the second fluid channel may or may not be in fluidic communication with each other. The first fluid channel may receive a sample fluid comprising one or more target analytes. The second fluid channel may receive an additional fluid from a source. The additional fluid may comprise a sheath fluid. The fluid inlets may each be oriented along a direction that is angled to a length of the fluidic channel with which they are in fluidic communication. The fluid inlets may have a cross sectional dimension that is the same as or different from the fluidic channel.

The microfluidic devices may comprise one or more fluid outlets. The fluid outlets may be in fluidic communication with the fluidic channel. The fluid outlets may each be oriented along a direction that is angled to a length of the fluidic channel with which they are in fluidic communication. The fluid outlets may have a cross sectional dimension that is the same as or different from the fluidic channel. The fluid outlets may comprise a first fluid outlet and a second fluid outlet. The first fluid outlet and the second fluid outlet may or may not be in fluidic communication with each other. The first fluid outlet may receive the target analytes separated from the fluid. The second fluid outlet may receive the remaining fluid (e.g., fluid absent at least a portion of the target analytes). The remaining fluid may flow in the microfluidic channel along the same direction as the original fluid (i.e., the fluid prior to separation).

Alternatively or additionally, the microfluidic devices may comprise an additional fluidic component in fluidic communication with the fluidic channel. The additional fluidic component may be configured to receive at least a portion of (e.g., greater than or equal to about 10%, 15%, 20%, 25%, 30%, 35%, 40%, 45%, 50%, 55%, 60%, 65%, 70%, 75%, 80%, 85%, 90%, 95% (mol %), or more) the target analytes separated from the fluid. The additional fluidic component may be used to remove the target analytes from the microfluidic devices. In some cases, the additional fluidic component is a tubing. The tubing may be a microtubing. The additional fluidic component may further be in communication with one or more sample inlets of detection, processing and/or analysis devices. The additional fluidic component may be configured to direct at least a portion of received target analytes into the detection, processing and/or analysis devices for detection, processing and/or analysis. In some cases, the additional fluidic component may be configured to remove large volume or quantity of separated analytes from the microfluidic chips. With the aid of the additional fluidic component, a microfluidic device as provided herein may be capable of processing large quantity of fluid samples or fluid samples having a large volume (a fluid sample having a volume that is greater than or equal to about 10 mL, 15 mL, 20 mL, 30 mL, 35 mL, 40 mL, 45 mL, 50 mL, 55 mL, 60 mL, 65 mL, 70 mL, 75 mL, 80 mL, 85 mL, 90 mL, 95 mL, 100 mL, or more). In some cases, a microfluidic device comprises a plurality of additional fluidic components in fluidic communication with the fluidic channel (e.g., a plurality of tubings with the same or different sizes).

In some cases, an additional fluidic channel may be comprised in the microfluidic device. The additional fluidic channel may be in fluidic communication with the fluidic channel. The additional fluidic channel may be configured to receive and retain at least a portion of the target analytes. The additional fluidic channel may comprise one or more obstacles disposed therein. The one or more obstacles may be an array of one or more obstacles. The one or more obstacles may or may not be oriented at a single direction. In some cases, the one or more obstacles are uniformed distributed within the additional fluidic channel. The one or more obstacles may be configured to capture the target analytes. The one or more obstacles may have a V-shaped or U-shaped configuration. Each of the one or more obstacles may comprise an opening. The opening may have a dimension that is configured to retain the captured target analytes. The opening may have a size that is greater than or equal to a size the target analytes. The one or more obstacles may be utilized to process samples having a small volume (e.g., a volume that is less than or equal to about 2,000 microliters (μL), 1,500 μL, 1,000 μL, 950 μL, 900 μL, 850 μL, 800 μL, 750 μL, 700 μL, 650 μL, 600 μL, 550 μL, 500 μL, 450 μL, 400 μL, 350 μL, 300 μL, 250 μL, 200 μL, 180 μL, 160 μL, 140 μL, 120 μL, 100 μL, 90 μL, 80 μL, 70 μL, 60 μL, 50 μL, 45 μL, 40 μL, 35 μL, 30 μL, 25 μL, 20 μL, 15 μL, 10 μL, 8 μL, 6 μL, 5 μL, 4 μL, 3 μL, 2 μL, 1 μL, or less). The one or more obstacles may facilitate separation and capturing of target analytes from a fluid sample which comprises the target analytes at a low concentration (e.g., target analytes has a concentration less than or equal to about 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05% (vol %, wt %, or mol %), or less). The one or more obstacles may facilitate separation and capturing of target analytes from a fluid sample which comprises a small number of the target analytes (e.g., a fluid which comprise less than or equal to about 50,000, 45,000, 40,000, 35,000, 30,000, 25,000, 20,000, 15,000, 10,000, 9,500, 9,000, 8,500, 8,000, 7,500, 7,000, 6,500, 6,000, 5,500, 5,000, 4,500, 4,000, 3,500, 3,000, 2,500, 2,000, 1,500, 1,000, 900, 800, 700, 600, 500 target analytes, or less).

As will be appreciated, the microfluidic devices may further comprise one or more fluidic pumps. The one or more fluidic pumps may be configured to transport fluidics within the microfluidic devices. The one or more fluidic pumps may be in fluidic communication with the fluidic channel, the fluid inlets, the fluid outlets, the additional fluidic channel, and/or any other components of the microfluidic device. The one or more fluidic pumps may comprise a plurality of valves.

In some aspects, a microfluidic device of the present disclosure may comprise a fluidic channel and one or more obstacles disposed therein. The one or more obstacles may be obstacles as described above or elsewhere herein. The one or more obstacles may be uniformly distributed within the fluidic channel. The one or more obstacles may comprise any number of individual obstacles, for example, greater than or equal to about 2, 3, 4, 5, 6, 7, 8, 9, 10, 11, 12, 13, 14, 15, 16, 17, 18, 19, 20, 22, 24, 26, 28, 30, 35, 40, 45, 50, 55, 60, 65, 70, 75, 80, 85, 90, 100, 110, 120, 130, 140, 150, 160, 170, 180, 190, 200 obstacles or more. The one or more obstacles may be an array of obstacles. The array of obstacles may or may not be oriented or aligned along a direction. The array of obstacles may be oriented at an angle relative to a direction of a fluid flow in the fluidic channel. The angle may be greater than 0°. The angle may be less than 90°. The angle may be any value that is greater than 0° and less than 90°, for example, between about 1° and 85°, or between 5° and 30°. The one or more obstacles may be configured to separate one or more target analytes (e.g., particles) from a fluid flowing through the fluidic channel. The fluid may be any fluids as described above or elsewhere herein. For example, the fluid may comprise biofluids including naturally occurring fluids (e.g., blood, sweat, tears, ear flow, sputum, lymph, bone marrow suspension, urine, saliva, semen, vaginal flow, cerebrospinal fluid, cervical lavage, brain fluid, ascites, milk, secretions of the respiratory, intestinal or genitourinary tract, amniotic fluid, and water samples), fluids into which cells have been introduced (e.g., culture media and liquefied tissue samples), or combinations thereof.

The target analytes may be any analytes that are of interest. The target analytes may be particles, such as biological particles as described above or elsewhere herein. For example, the target analytes may comprise any cells or components thereof, viruses, bacteria, proteins, carbohydrates, nucleic acid molecules (such as deoxyribonucleic acid (DNA), ribonucleic acid (RNA)), lipid, or combinations thereof. Non-limiting examples of cells may include, tumor cells, red blood cells, white blood cells (such as T cells, B cells, and helper T cells), infected cells, trophoblasts, fibroblasts, stem cells, epithelial cells, infectious organisms (e.g., bacteria, protozoa, and fungi), cancer cells, bone marrow cells, fetal cells, progenitor cells, foam cells, mesenchymal cells, immune system cells, endothelial cells, endometrial cells, connective tissue cells, trophoblasts, bacteria, fungi, or pathogens, or combinations thereof.

In some cases, cells may comprise senescent cells. The senescent cells may comprise senescent tumor cells. Senescent tumor cells may comprise tumor cells that are benign or malignant. Non-limiting examples of tumors may include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastrointestinal system carcinomas, colon carcinoma, pancreatic cancer, breast cancer, genitourinary system carcinomas, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, endocrine system carcinomas, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, or combinations thereof. The tumors may be associated with various types of organs. Non-limiting examples of organs may include brain, breast, liver, lung, kidney, prostate, ovary, spleen, lymph node (including tonsil), thyroid, pancreas, heart, skeletal muscle, intestine, larynx, esophagus, stomach, or combinations thereof. The obstacles (e.g., an array of obstacles) may separate one or more senescent cells from a fluid upon flow of the fluid through the microfluidic channel. The fluid may comprise one or more non-senescent cells, in addition to the senescent cells. The obstacles may separate the one or more senescent cells from the non-senescent cells while the fluid flows through the fluidic channel. The obstacles may separate the senescent cells at a high efficiency (e.g., at an efficiency greater than about 50%, 55%, 60%, 65%, 70%, 72%, 74%, 76%, 78%, 80%, 82%, 84%, 86%, 88%, 90%, 91%, 92%, 93%, 94%, 95%, 96%, 97%, 98%, 99%, or more).

The target analytes can be of any size, shape, or geometry. The target analytes may have an average size that is greater than or equal to about 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, or more. In some cases, the target analytes have an average size that falls between any of the two values described above or elsewhere herein, for example, between about 15 μm and 30 μm.

The one or more obstacles may be configured to separate one or more target analytes with a high throughput. The microfluidic device of the present disclosure may be configured to process a fluid sample having a volume that is greater than or equal to about 10 mL, 15 mL, 20 mL, 30 mL, 35 mL, 40 mL, 45 mL, 50 mL, 55 mL, 60 mL, 65 mL, 70 mL, 75 mL, 80 mL, 85 mL, 90 mL, 95 mL, 100 mL, or more. In cases where a large quantity of sample is to be processed or multiplex assaying is desired, the system of the present disclosure may comprise a plurality of microfluidic devices, e.g., greater than or equal to about 2, 3, 4, 5, 6, 7, 8, 9, 10, 11, 12, 13, 14, 15, 16, 17, 18, 19, 20, 25, 30 devices or more. The plurality of devices may or may not be in fluidic communication with one another. The plurality of devices may be in fluidic communication with one or more common fluid inlets and/or outlets. The plurality of devices may be arranged in parallel, in series or in a combined configuration of in series and in parallel. In some examples, individual devices of the plurality of devices may be stacked in vertical direction (or a direction perpendicular to a plane within which the fluidic channel is disposed. Individual devices of the plurality of devices may or may not be the same in terms of size, shape, geometry, sample processing capability, and/or obstacles (e.g., number of obstacles, shape, size, dimension, geometry, arrangement of the obstacles) comprised in the fluidic channel.

In some examples, a single microfluidic device may comprise multiple fluidic channels (e.g., greater than or equal to about 2, 3, 4, 5, 6, 7, 8, 9, 10, 12, 14, 16, 18, 20, 25, 30, 35, 40, 45, 50 fluidic channels or more). The fluidic channels may or may not be in fluidic communication with one another. The fluidic channels may be arranged in parallel, in series or in combined configuration of in parallel and in series. The fluidic channels may each comprise one or more obstacles (e.g., an array of obstacles). Obstacles comprised in different fluidic channels may be the same or may be different. Obstacle arrays may differ from one another in number of obstacles comprised in the array, size, dimension, shape, geometry, cross sections, configuration of obstacles, spacing size between adjacent obstacles of the array, and/or arrangement of obstacles in the array. The fluidic channels may be configured to process the same sample. The fluidic channels may each be configured to process a different sample. The fluidic channels may each be configured to separate a different type of target analytes from a fluid sample. It should be noted that the disclosure is not limited to the various examples described above and elsewhere herein. For example, in some cases, instead of having multiple microfluidic devices or a single device having multiple channels, various types of target analytes may be separated from a fluid using a microfluidic device comprising a fluidic channel which comprises multiple sections along a direction of fluid flow. Each sections of the fluidic channel may comprise a different array of obstacles which is configured to separate, isolate and/or capture a given type of analytes.

The target analytes may be separated with a high efficiency when the fluid is directed to flow through the fluidic channel at a given flow rate. As provided herein, the flow rate may be greater than or equal to about 100 milliliters/hour (mL/hr), 120 mL/hr, 140 mL/hr, 160 mL/hr, 180 mL/hr, 200 mL/hr, 220 mL/hr, 240 mL/hr, 260 mL/hr, 280 mL/hr, 300 mL/hr, 320 mL/hr, 340 mL/hr, 360 mL/hr, 380 mL/hr, 400 mL/hr, 420 mL/hr, 440 mL/hr, 460 mL/hr, 480 mL/hr, 500 mL/hr, 550 mL/hr, 600 mL/hr, 650 mL/hr, 700 mL/hr, 750 mL/hr, 800 mL/hr, 850 mL/hr, 900 mL/hr, 950 mL/hr, 1,000 mL/hr, or more. In some cases, the flow rate is between any of the two values described above and elsewhere herein, for example, about 250 mL/hr.

The separation efficiency may be determined as a percentage (e.g., number or mole percent) of original target analytes comprised in the fluid that is separated from the fluid by the obstacles. For example, upon flow of a fluid comprising 10,000 particles through the fluidic channel, if 5,000 particles are separated or isolated from the fluid, then the efficiency is 50%. In another example, if 70 mol % of the target analytes that are originally comprised in a fluid is separated from the fluid as the fluid flows through the fluidic channel, then the efficiency is 70%. As provided herein, the target analytes may be separated from the fluid with a high efficiency. The efficiency may be greater than or equal to about 50%, 55%, 60%, 65%, 70%, 72%, 74%, 76%, 78%, 80%, 82%, 84%, 86%, 88%, 90%, 91%, 92%, 93%, 94%, 95%, 96%, 97%, 98%, 99%, or more. In some cases, the target analytes are separated from the fluid at an efficiency that falls between any of the two values described above or elsewhere herein, for example, about 75%.

In some aspects, the systems of the present disclosure comprise a microfluidic device which may separate target analytes from small sample volumes. The microfluidic device may comprise a fluidic channel. The fluidic channel may comprise one or more obstacles disposed therein. The obstacles may be any obstacles as described above or elsewhere herein. The obstacles may comprise microstructures, nanostructures or combinations thereof. At least a subset of the obstacles is nonporous. In some cases, all of the obstacles are nonporous. The obstacles may be 3D structures. The obstacles may have openings in x-, y- and z-directions. The obstacles may deform when experiencing a pressure. An average spacing size between adjacent obstacles may vary. The average spacing size may be adjusted depending upon a variety of factors, including such as dimension of the microfluidic channel, number of obstacles disposed in the microfluidic channel, sample volume, sizes, dimensions, geometries of target analytes, fluid flow rate, or combinations thereof. In some cases, the obstacles may have an average spacing size greater than or equal to about 10 nanometers (nm), 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1 μm, 5 μm, 10 μm, 20 μm, 30 μm, 40 μm, 50 μm, 60 μm, 70 μm, 80 μm, 90 μm, 100 μm, or more. In some cases, the average spacing size may be less than or equal to about 200 μm, 180 μm, 160 μm, 140 μm, 120 μm, 100 μm, 85 μm, 75 μm, 65 μm, 55 μm, 45 μm, 35 μm, 25 μm, 15 μm, 5 μm, 1 μm, 850 nm, 750 nm, 650 nm, 550 nm, 450 nm, 350 nm, 250 nm, 150 nm, 100 nm, or less. In some cases, the average spacing size may be any of the values described above or elsewhere herein, for example, between about 1 μm and 100 μm.

Surfaces of the obstacles may be modified. For example, the obstacles may be coated with chemical or biological reagents, e.g., a charged moiety, an antibody. The obstacles may be treated with reagents such that they may bind specifically to a given type of target analytes. Non-limiting examples of reagents that may be used for treating, modifying the obstacles include polymers, carbohydrates, a molecule that binds to a cell surface receptor, an oligo- or polypeptide, a viral or bacterial protein, a nucleic acid, or a carbohydrate that binds a population of cells, or combinations thereof.

The one or more obstacles may comprise an array of obstacles. The obstacles may be configured to separate one or more target analytes from a fluid having a small volume upon flow of the fluid through the fluidic channel. The target analytes may comprise any analytes as described above or elsewhere herein, for example, biological particles. In some cases, the target analytes comprise senescent cells.

The fluid may have a volume that is less than or equal to about 2,000 microliters (μL), 1,500 μL, 1,000 μL, 950 μL, 900 μL, 850 μL, 800 μL, 750 μL, 700 μL, 650 μL, 600 μL, 550 μL, 500 μL, 450 μL, 400 μL, 350 μL, 300 μL, 250 μL, 200 μL, 180 μL, 160 μL, 140 μL, 120 μL, 100 μL, 90 μL, 80 μL, 70 μL, 60 μL, 50 μL, 45 μL, 40 μL, 35 μL, 30 μL, 25 μL, 20 μL, 15 μL, 10 μL, 8 μL, 6 μL, 5 μL, 4 μL, 3 μL, 2 μL, 1 μL, or less. In some cases, the fluid has a volume that is between any of the two values described above and elsewhere herein, for example, between about 1 μL and 500 μL.

The fluid may comprise a small number of target analytes. For example, the fluid may comprise less than or equal to about 50,000, 45,000, 40,000, 35,000, 30,000, 25,000, 20,000, 15,000, 10,000, 9,500, 9,000, 8,500, 8,000, 7,500, 7,000, 6,500, 6,000, 5,500, 5,000, 4,500, 4,000, 3,500, 3,000, 2,500, 2,000, 1,500, 1,000, 900, 800, 700, 600, 500 target analytes, or less. In some cases, the number of target analytes comprised in the fluid may be between any of the two values describe above or elsewhere herein, for example, between about 1,000 and about 20,000.

In some cases, the fluid may comprise target analytes at a low concentration. For example, the fluid may comprise the target analytes at a concentration less than or equal to about 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05% (vol %, wt %, or mol %), or less. In some cases, the target analytes have a concentration between any of two values describe above or elsewhere herein, for example, between about 1% and about 10%.

As provided herein, the microfluidic devices may be monolithic, or may be fabricated in one or more components which may be assembled. Various components or layers of the devices may be assembled or bonded together using various methods or tools including e.g., adhesives, clamps, heat, anodic heating, or reactions.

### Methods

Also provided herein are methods for separating, isolating, detecting, and/or analyzing target analytes such as biological particles. Such methods may be used for processing senescence cells.

In an aspect, a method may comprise directing a fluid comprising one or more target analytes into a microfluidic device. The microfluidic device may be any microfluidic devices described above or elsewhere herein. For example, the microfluidic device may comprise a fluidic channel and one or more obstacles disposed therein. The obstacles may be any obstacles of the present disclosure. The one or more obstacles may be an array of obstacles. The array of obstacles may be oriented or aligned along a certain direction. The direction along which the obstacle array is aligned may be angled relative to a direction of fluid flow in said fluidic channel. The direction of fluid flow may be a direction along which the fluid comprising the target analytes flows within the fluidic channel. The direction of fluid flow may not change as the fluid flows through the fluidic channel. There may be an angle between the direction along which the array of obstacles is aligned and the direction of the fluid flow. The angle may be an oblique angle. The angle may be between about 0° and about 90°. In some cases, the angle may be greater than about 0°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85° or more. In some cases, the angle may less than about 90°, 85°, 80°, 75°, 70°, 65°, 60°, 55°, 50°, 45°, 40°, 35°, 30°, 25°, 20°, 15°, 10°, 9°, 7°, 5°, 3°, 1°, or less. In some cases, the angle may be between any of the values described above or elsewhere herein, for example, between about 0° and 20°. In some cases, all of the obstacles are angled relative to the direction of the fluid flow.

Next, the fluid comprising the target analytes is directed to flow through the fluid channel. Upon flow of the fluid through the fluidic channel, at least a portion of the target analytes may be separated from the fluid with the aid of the obstacles. The obstacles may be configured to direct some or all of the target analytes that are separated from the fluid to flow along or towards a direction which differs from the direction of fluid flow. As described above or elsewhere herein, the obstacles may separate the target analytes based at least partially on sizes of the target analytes. The obstacles may have an average spacing size which may permit analytes having an average size below a threshold value to pass through while hinder the movement of analytes having an average size equal to or above the threshold value. The threshold value may or may not be average spacing size of the obstacles. The threshold value may be determined using reference analytes (e.g., reference particles having known sizes). In some cases, the average spacing size may be adjusted for separating different types of target analytes. The adjustment may be achieved by removing, adding and/or substituting one or more obstacles disposed in the fluidic channel. For example, one or more obstacles may be removed from the fluidic channel to increase an average spacing size of the obstacles. Similarly, in cases where a smaller average spacing size is desired, one or more obstacles may be added to the fluidic channel. In some cases, the average spacing size may be altered by substituting one or more obstacles with different types of obstacles, e.g., obstacles with different cross sections, dimensions, geometries etc.

As described above and elsewhere herein, a distance may exist between the array of obstacles and a side wall of the microfluidic channel. In some cases, at least one obstacle disposed in the microfluidic channel is adjacent to a side wall of the microfluidic channel. For example, a distance between at least one obstacle disposed in the microfluidic channel and a side wall of the channel may be less than or equal to about 1 μm, 0.5 μm, 0.4 μm, 0.3 μm, 0.2 μm, 0.1 μm, 0.05 μm, or less. The distance between the array of obstacles and a side wall of the microfluidic channel may increase along a direction of fluid flow.

The target analytes may be any types of target analytes as described above or elsewhere herein. For example, the target analytes may be biological particles. For example, the target analytes may comprise any cells or components thereof, viruses, bacteria, proteins, carbohydrates, nucleic acid molecules (such as deoxyribonucleic acid (DNA), ribonucleic acid (RNA)), lipid, or combinations thereof. Non-limiting examples of cells may include, tumor cells, red blood cells, white blood cells (such as T cells, B cells, and helper T cells), infected cells, trophoblasts, fibroblasts, stem cells, epithelial cells, infectious organisms (e.g., bacteria, protozoa, and fungi), cancer cells, bone marrow cells, fetal cells, progenitor cells, foam cells, mesenchymal cells, immune system cells, endothelial cells, endometrial cells, connective tissue cells, trophoblasts, bacteria, fungi, or pathogens, or combinations thereof. In some cases, cells may comprise senescent cells. The senescent cells may comprise senescent tumor cells.

In some cases, the method further comprises directing an additional fluid into the microfluidic device. The additional fluid may or may not the same as the fluid that comprises the target analytes. The additional fluid and the fluid may be miscible, partially miscible or immiscible. The additional may comprise a sheath fluid, e.g., a buffer. The additional fluid may be used to ensure that the fluid is flowing along or towards a certain direction (e.g., the direction of fluid flow).

While the target analytes are separated from the fluid, at least a portion (e.g., greater than or equal to about 20%, 25%, 30%, 35%, 40%, 45%, 50%, 55%, 60%, 65%, 70%, 75%, 80%, 85%, 90%, 95%, or more) of the separated target analytes may be captured. The target analytes may be captured by one or more obstacles disposed in a fluidic component (e.g., an additional fluid channel) comprised in the microfluidic device. The additional fluidic channel may be in fluidic communication with the fluidic channel. The one or more obstacles may or may not be the same as the obstacles disposed in the fluidic channel. The one or more obstacles may be an array of obstacles. The one or more obstacles may be randomly or uniformly distributed in the additional fluidic channel. The one or more obstacles may have a V-shaped pattern. Each of the one or more obstacles may comprise an opening. The opening may have a dimension that is configured to retain the captured target analytes. The opening may have a size that is greater than or equal to a size the target analytes.

In some cases, the separated target analytes may be detected. In some cases, the separated target analytes may be removed without any further analyses. In some cases, the separated target analytes may be directed to one or more detection and/or analysis units for detection and/or analyses.

As provided herein, the methods of the present disclosure may separate or isolate target analytes from a fluid at a high sensitivity. The sensitivity may be determined as a ratio of (i) target analytes separated from the fluid to (ii) a total of target analytes and non-target analytes separated from the fluid. For example, if 50% of the analytes separated from the fluid are target analytes, then the sensitivity is 50%. The methods of the present disclosure may separate or isolate target analytes at a sensitivity greater than or equal to about 60%, 65%, 70%, 75%, 80%, 82%, 84%, 86%, 88%, 90%, 91%, 92%, 93%, 94%, 95%, 96%, 97%, 98%, 99%, or more.

The methods of the present disclosure may separate or isolate target analytes from a fluid at a high specificity. The specificity may be determined as a ratio of (i) non-target analytes remained in (or not separated from) the fluid to (ii) a total of target analytes and non-target analytes remained in (or not separated from) the fluid. As an example, if 50% of the analytes remained in the fluid are non-target analytes, then the specificity is 50%. The methods of the present disclosure may separate or isolate target analytes at a specificity greater than or equal to about 60%, 65%, 70%, 75%, 80%, 82%, 84%, 86%, 88%, 90%, 91%, 92%, 93%, 94%, 95%, 96%, 97%, 98%, 99%, or more.

In some aspects of the present disclosure, the methods may comprise directing a fluid comprising one or more target analytes into a microfluidic device. The microfluidic device may be any microfluidic devices described above or elsewhere herein. For example, the microfluidic device may comprise a fluidic channel and one or more obstacles disposed therein. The one or more obstacles may be obstacles as described above or elsewhere herein. The one or more obstacles may be uniformly distributed within the fluidic channel. The one or more obstacles may comprise any number of individual obstacles, for example, greater than or equal to about 2, 3, 4, 5, 6, 7, 8, 9, 10, 11, 12, 13, 14, 15, 16, 17, 18, 19, 20, 22, 24, 26, 28, 30, 35, 40, 45, 50, 55, 60, 65, 70, 75, 80, 85, 90, 100, 110, 120, 130, 140, 150, 160, 170, 180, 190, 200 obstacles or more. The one or more obstacles may be an array of obstacles. The array of obstacles may or may not be oriented or aligned along a single direction. The one or more obstacles may be configured to separate one or more target analytes (e.g., particles) from a fluid flowing through the fluidic channel. The fluid may be any fluids as described above or elsewhere herein. For example, the fluid may comprise biofluids.

Next, the fluid may be directed to flow through the fluidic channel. Upon flow of the fluid through the fluidic channel, at least a portion of the target analytes may be separated from the fluid using the one or more obstacles. The methods may separate the target analytes with a high efficiency while the fluid is directed to flow through the fluidic channel at a given flow rate. For example, the fluid may be directed through the fluidic channel at a flow rate greater than or equal to about 100 milliliters/hour (mL/hr), 120 mL/hr, 140 mL/hr, 160 mL/hr, 180 mL/hr, 200 mL/hr, 220 mL/hr, 240 mL/hr, 260 mL/hr, 280 mL/hr, 300 mL/hr, 320 mL/hr, 340 mL/hr, 360 mL/hr, 380 mL/hr, 400 mL/hr, 420 mL/hr, 440 mL/hr, 460 mL/hr, 480 mL/hr, 500 mL/hr, 550 mL/hr, 600 mL/hr, 650 mL/hr, 700 mL/hr, 750 mL/hr, 800 mL/hr, 850 mL/hr, 900 mL/hr, 950 mL/hr, 1,000 mL/hr, or more. In some cases, the flow rate is between any of the two values described above and elsewhere herein, for example, about 250 mL/hr.

The separation efficiency may be determined as a percentage (e.g., number or mole percent) of original target analytes comprised in the fluid that is separated from the fluid by the obstacles. For example, upon flow of a fluid comprising 10,000 particles through the fluidic channel, if 5,000 particles are separated or isolated from the fluid, then the efficiency is 50%. In another example, if 70 mol % of the target analytes that are originally comprised in a fluid is separated from the fluid as the fluid flows through the fluidic channel, then the efficiency is 70%. As provided herein, the target analytes may be separated from the fluid with an efficiency greater than or equal to about 50%, 55%, 60%, 65%, 70%, 72%, 74%, 76%, 78%, 80%, 82%, 84%, 86%, 88%, 90%, 91%, 92%, 93%, 94%, 95%, 96%, 97%, 98%, 99%, or more. In some cases, the target analytes are separated from the fluid at an efficiency that falls between any of the two values described above or elsewhere herein, for example, about 75%.

Some aspects of the present disclosure provide a method for separating one or more target analytes from a fluid sample having a small volume. The method may comprise directing a fluid having a small volume into a microfluidic device. The microfluidic device may be any microfluidic devices as described above or elsewhere herein. For example, the microfluidic device may comprise a fluidic channel which may comprise one or more obstacles disposed therein. The obstacles may be any obstacles described above or elsewhere herein. In some example, the obstacles may be an array of obstacles.

The fluid may have a volume that is less than or equal to about 2,000 microliters (μL), 1,500 μL, 1,000 μL, 950 μL, 900 μL, 850 μL, 800 μL, 750 μL, 700 μL, 650 μL, 600 μL, 550 μL, 500 μL, 450 μL, 400 μL, 350 μL, 300 μL, 250 μL, 200 μL, 180 μL, 160 μL, 140 μL, 120 μL, 100 μL, 90 μL, 80 μL, 70 μL, 60 μL, 50 μL, 45 μL, 40 μL, 35 μL, 30 μL, 25 μL, 20 μL, 15 μL, 10 μL, 8 μL, 6 μL, 5 μL, 4 μL, 3 μL, 2 μL, 1 or less. In some cases, the fluid has a volume that is between any of the two values described above and elsewhere herein, for example, between about 1 μL and 500 μL.

The fluid may comprise a small number of target analytes. For example, the fluid may comprise less than or equal to about 50,000, 45,000, 40,000, 35,000, 30,000, 25,000, 20,000, 15,000, 10,000, 9,500, 9,000, 8,500, 8,000, 7,500, 7,000, 6,500, 6,000, 5,500, 5,000, 4,500, 4,000, 3,500, 3,000, 2,500, 2,000, 1,500, 1,000, 900, 800, 700, 600, 500 target analytes, or less. In some cases, the number of target analytes comprised in the fluid may be between any of the two values describe above or elsewhere herein, for example, between about 1,000 and about 20,000.

In some cases, the fluid may comprise target analytes at a low concentration. For example, the fluid may comprise the target analytes at a concentration less than or equal to about 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05% (vol %, wt %, or mol %), or less. In some cases, the target analytes have a concentration between any of two values describe above or elsewhere herein, for example, between about 1% and about 10%.

As provided above or elsewhere herein, the target analytes may be any analytes that are of interest. The target analytes may be particles, such as biological particles as described above or elsewhere herein. In some examples, the target analytes may comprise any cells or components thereof, viruses, bacteria, proteins, carbohydrates, nucleic acid molecules (such as deoxyribonucleic acid (DNA), ribonucleic acid (RNA)), lipid, or combinations thereof. Non-limiting examples of cells may include, tumor cells, red blood cells, white blood cells (such as T cells, B cells, and helper T cells), infected cells, trophoblasts, fibroblasts, stem cells, epithelial cells, infectious organisms (e.g., bacteria, protozoa, and fungi), cancer cells, bone marrow cells, fetal cells, progenitor cells, foam cells, mesenchymal cells, immune system cells, endothelial cells, endometrial cells, connective tissue cells, trophoblasts, bacteria, fungi, or pathogens, or combinations thereof.

In some cases, cells may comprise senescent cells. The senescent cells may comprise senescent tumor cells. Senescent tumor cells may comprise tumor cells that are benign or malignant. Non-limiting examples of tumors may include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastrointestinal system carcinomas, colon carcinoma, pancreatic cancer, breast cancer, genitourinary system carcinomas, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, endocrine system carcinomas, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, or combinations thereof. The tumors may be associated with various types of organs. Non-limiting examples of organs may include brain, breast, liver, lung, kidney, prostate, ovary, spleen, lymph node (including tonsil), thyroid, pancreas, heart, skeletal muscle, intestine, larynx, esophagus, stomach, or combinations thereof. In some cases, the target analytes may comprise senescent T cells, senescent cells of different kinds of white blood cells, senescent microphages, senescent lung, breast, colon, prostate, gastric, hepatic, ovarian, esophageal, or bronchial epithelial or stromal cells, senescent skin epithelial or stromal cells, senescent glial cells, senescent vascular endothelial or stromal cells, or combinations thereof. The obstacles (e.g., an array of obstacles) may separate one or more senescent cells from a fluid upon flow of the fluid through the microfluidic channel. The fluid may comprise one or more non-senescent cells, in addition to the senescent cells. The obstacles may separate the one or more senescent cells from the non-senescent cells while the fluid flows through the fluidic channel. The obstacles may separate the senescent cells at a high efficiency (e.g., at an efficiency greater than about 50%, 55%, 60%, 65%, 70%, 72%, 74%, 76%, 78%, 80%, 82%, 84%, 86%, 88%, 90%, 91%, 92%, 93%, 94%, 95%, 96%, 97%, 98%, 99%, or more).

The target analytes can be of any size, shape, or geometry. The target analytes may have an average size that is greater than or equal to about 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, or more. The target analytes may have an average size that is less than or equal to about 50 μm, 45 μm, 40 μm, 35 μm, 30 μm, 28 μm, 26 μm, 24 μm, 22 μm, 20 μm, 18 μm, 16 μm, 14 μm, 12 μm, 10 μm, 9 μm, 8 μm, 7 μm, 6 μm, 5 μm, 4 μm, 3 μm, 1 μm, 1 μm, or less. In some cases, the target analytes have an average size that falls between any of the two values described above or elsewhere herein, for example, between about 15 μm and 30 μm.

The method may further comprise directing the fluid to flow through the fluidic channel. Upon flow of the fluid through the fluidic channel, at least a portion of the target analytes may be separated or removed from the fluid using the obstacles. The method may separate at least about 60%, 65%, 70%, 75%, 80%, 85%, 90%, 95% of the target analytes, or more. The method may separate from the fluid less than or equal to about 30%, 25%, 20%, 18%, 16%, 14%, 12%, 10%, 8%, 6%, 4%, 2%, 1% of non-target analytes. In some cases, the obstacles may separate the target analytes by causing the target analytes to flow at or towards a direction which is different from a direction of the fluid in the fluidic channel. The direction of the fluid before and after removal of the target analytes may remain unchanged. During or after the separation, the target analytes and the fluid having at least a portion of the target analytes removed therefrom may flow out of the fluidic channel along different directions. For example, the separated target analytes and the fluid having at least a portion of the target analytes removed therefrom may be directed to a first fluid outlet and a second fluid outlet, respectively.

The target analytes separated from the fluid may be captured. The target analytes may be captured using one or more obstacles disposed in a fluidic component (e.g., an additional fluidic channel) of the microfluidic device. The one or more obstacles may or may not be the same as the obstacles disposed in the fluidic channel. The one or more obstacles used to capture the target analytes may be an array of capture obstacles. The one or more obstacles may be randomly or uniformly distributed in the additional fluidic channel. The one or more obstacles may have a V-shaped, or U-shaped pattern. Each of the one or more obstacles may comprise an opening. The opening may have a dimension that is configured to retain the captured target analytes. The opening may have a size that is greater than or equal to a size the target analytes.

The method may separate or isolate target analytes from a fluid at a high sensitivity. The sensitivity may be determined as a ratio of (i) target analytes separated from the fluid to (ii) a total of target analytes and non-target analytes separated from the fluid. For example, if 50% of the analytes separated from the fluid are target analytes, then the sensitivity is 50%. The methods of the present disclosure may separate or isolate target analytes at a sensitivity greater than or equal to about 60%, 65%, 70%, 75%, 80%, 82%, 84%, 86%, 88%, 90%, 91%, 92%, 93%, 94%, 95%, 96%, 97%, 98%, 99%, or more.

The methods of the present disclosure may separate or isolate target analytes from a fluid at a high specificity. The specificity may be determined as a ratio of (i) non-target analytes remained in (or not separated from) the fluid to (ii) a total of target analytes and non-target analytes remained in (or not separated from) the fluid. As an example, if 50% of the analytes remained in the fluid are non-target analytes, then the specificity is 50%. The methods of the present disclosure may separate or isolate target analytes at a specificity greater than or equal to about 60%, 65%, 70%, 75%, 80%, 82%, 84%, 86%, 88%, 90%, 91%, 92%, 93%, 94%, 95%, 96%, 97%, 98%, 99%, or more.

The target analytes may be detected during and/or after separation of the target analytes from the fluid. The detection may be performed in real-time while the separation is taking place. The detection may be performed at multiple time points while the separation is taking place. The detection may be performed subsequent to separation of the target analytes from the fluid. The detection may be performed on the microfluidic device. The detection may be performed after removing the target analytes from the microfluidic device. The detection may comprise detecting a presence or absence of the target analytes. The detection may comprise detecting an amount of the target analytes. The detection may comprise detecting a signal from the target analytes. The signal may be an optical signal. The optical signal may be an optical signal of any wavelength or frequency. The optical signal may comprise visible light, ultraviolet light and/or infrared light. The optical signal may be luminescent signals (e.g., bioluminescence, chemiluminescence, fluorescence). The signal may be an electrical signal. The electrical signals may comprise electrical current, voltage, impedance, resistance, capacitance, and/or conductance. Various techniques may be used for detecting target analytes, e.g., techniques from molecular biology (including recombinant techniques), cell biology (e.g., cell counting using a counting chamber (hemocytometer), plating methods, spectrophotometry, spectrometry (e.g., mass spectrometry), flow cytometry, Coulter counter etc.), immunoassay technology, microscopy (e.g., optical microscopy, fluorescent microscopy), image analysis, analytical chemistry, or combinations thereof.

In some cases, target analytes comprise one or more agents or moieties that may facilitate the detection. For example, the target analyets may comprise agents that may produce signals (e.g., light or electrical signals). The agents may be associated with or bind to the target analytes. The agents may specifically bind to a particular type of target analytes. In some cases, the agents are antibodies that bind to a cell surface protein. The antibodies may comprise one or more detection agents which may produce signals, e.g., detection agents that may emit, scatter, reflect, deflect or diffract light signals. In some examples, the target analytes may be treated (e.g., mixed) with one or more reagents. The treatment may occur prior to, during or after the separation is taking place. The one or more reagents may comprise stains. The stains may be any dye (e.g., a fluorescent dye), probe, substrate, or any chemical or biological substance that is suitable for staining a target analyte (e.g., a biological cell) or a portion thereof. The stains may enhance contrast and highlight structures of a stained object or a portion thereof. The stains may have a preference or specificity for a particular type of target analytes (e.g., a particular type of biological cells). In some cases, the stains mark (or stain) a given type of target analytes (or a portion thereof) in a particular color or fluorescence that is at least about 2, 3, 4, 5, 6, 7, 8, 9, 10 times greater in intensity than a staining intensity to another type of target analyets (or a portion thereof) at that same color or fluorescence spectrum.

The target analytes may be detected at a single molecule resolution. As an example, when the target analytes comprise cells such as senescent cells, the cells may be detected at a single cell resolution. The method may further comprise directing at least a portion of the target analytes from the microfluidic device to one or more analysis units for further analyses.

### Computer Systems

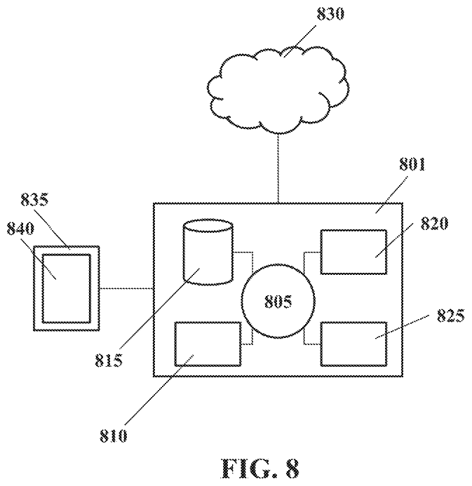

The present disclosure provides computer systems that are programmed to implement methods of the disclosure. FIG. 8 shows a computer system 801 that is programmed or otherwise configured to perform various methods of the present disclosure. The computer system 801 can regulate various aspects of methods and systems of the present disclosure, such as, for example, regulating fluid flow in a microfluidic device, adjusting flow rate of a fluid within a microfluidic device, directing a fluid to, from and/or through a microfluidic device. The computer system 801 can be an electronic device of a user or a computer system that is remotely located with respect to the electronic device. The electronic device can be a mobile electronic device.

The computer system 801 includes a central processing unit (CPU, also “processor” and “computer processor” herein) 805, which can be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 can be a data storage unit (or data repository) for storing data. The computer system 801 can be operatively coupled to a computer network (“network”) 830 with the aid of the communication interface 820. The network 830 can be the Internet, an internet and/or extranet, or an intranet and/or extranet that is in communication with the Internet. The network 830 in some cases is a telecommunication and/or data network. The network 830 can include one or more computer servers, which can enable distributed computing, such as cloud computing. The network 830, in some cases with the aid of the computer system 801, can implement a peer-to-peer network, which may enable devices coupled to the computer system 801 to behave as a client or a server.

The CPU 805 can execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and writeback.

The CPU 805 can be part of a circuit, such as an integrated circuit. One or more other components of the system 801 can be included in the circuit. In some cases, the circuit is an application specific integrated circuit (ASIC).

The storage unit 815 can store files, such as drivers, libraries and saved programs. The storage unit 815 can store user data, e.g., user preferences and user programs. The computer system 801 in some cases can include one or more additional data storage units that are external to the computer system 801, such as located on a remote server that is in communication with the computer system 801 through an intranet or the Internet.

The computer system 801 can communicate with one or more remote computer systems through the network 830. For instance, the computer system 801 can communicate with a remote computer system of a user (e.g., a lab technician, a physician). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 801 via the network 830.

Methods as described herein can be implemented by way of machine (e.g., computer processor) executable code stored on an electronic storage location of the computer system 1101, such as, for example, on the memory 810 or electronic storage unit 815. The machine executable or machine readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 can include or be in communication with an electronic display 835 that comprises a user interface (UI) 840 for providing, for example, parameters and/or information of microfluidic devices, or instructions for handling one or more samples. Examples of UP s include, without limitation, a graphical user interface (GUI) and web-based user interface.

Methods and systems of the present disclosure can be implemented by way of one or more algorithms. An algorithm can be implemented by way of software upon execution by the central processing unit 1105. The algorithm can, for example, perform methods of the present disclosure.

### Examples

Chips and manufacturing thereof. The Polydimethylsiloxane (PDMS) microfluidic channel was fabricated with soft lithography. The mask was designed with the AutoCAD software (Autodesk Inc., San Rafael, Calif.) and produced by Photo Sciences, Inc. (Torrance, Calif.). The silicon master as a PDMS mold was produced by standard photolithography and deep reactive ion etching (DRIE) techniques. To fabricate the PDMS mold, 2 μm g-line photoresist (FujiFilm, USA) was coated on a 6-inch silicon wafer by spin coating. Followed with UV exposure to transfer the pattern from a mask to the photoresist layer, the silicon wafer was developed to generate a pattern on photoresist. After hard-bake, the wafer was etched by DRIE to produce channels with the desired depth. For the chip, the channel depth was controlled between 30 and 35 μm. Finally, a Teflon layer was deposited on all surfaces of the silicon wafer to ensure a smooth PDMS peeling-off process. The chip was prepared by bonding the PDMS-replica channel onto a glass slide after treated with plasma (PDC-001, Harrick Plasma, USA). For the parallel-processing chip, five layers of identical PDMS channels were stacked up with the inlets and outlets aligned along the vertical direction. Before use, the device was incubated at 100° C. overnight to prevent the fluid leakage and confirmed by the flow-through of 1×PBS buffer. Microtubing with 0.8 mm ID and 1.4 mm OD (Cole-Parmer, USA) was connected to the PDMS channels for fluid delivery. The devices were disposed after each run of biological samples.

Assembly and quality control of a chip platform. An epifluorescence microscope (IX83, Olympus, Japan) connected with a CCD camera (QIClick, QImaging, Canada) was used to observe and record the cell separation process inside the microfluidic channel. The blood sample and 1×PBS buffer with 0.05% BSA were stored in 3 mL and 50 mL syringes (BD Biosciences, USA), respectively. A 0.45 μm syringe filter (Acrodisc, Pall Life Sciences, USA) was connected to the syringe storing the PBS buffer to prevent contaminant from flowing into the microchannel and clogging the filter array (or obstacle array). Two infusion syringe pumps (NE-1600, New Era Pump Systems, USA; and KDS 100, KD Scientific, USA) were used to control the flow rates. When the chip was tested with cells spiked in the undiluted whole blood, the buffer flow rate was usually 3 times of that of the blood sample. This ratio was decreased to 2 when the chip was tested with low concentration of cells or beads solution alone. Due to the sedimentation of cells, the syringe containing blood sample was vertically positioned to ensure the most of the cells flowed into the microtubing. When the chip contains only one outlet with capture arrays for cell trapping, a much longer microtubing was connected to the other outlet without capture arrays to balance the hydrodynamic resistance through both outlets. Before separation, 1×PBS buffer with 0.05% BSA flowed through the microchannel and microtubing for ˜15 min to remove any remaining air bubbles and reduce nonspecific bonding to the channels. For the portable detection of MSCs on the chip, an iPhone 6 smart phone connected with a 60-100× mobile phone microscope lens (Neewer, China) was used to take and visualize the images.

Preparation of samples for validating chips. Fresh human whole blood from healthy donors collected within 24 h was purchased from AllCells Inc. (Alameda, Calif.). The blood samples were collected with K2EDTA blood collection tubes (BD Biosciences, USA). Polystyrene beads with varied sizes were purchased from Polysciences, Inc. (Warminster, Pa.) and Bangs Laboratories, Inc (Fishers, Ind.). Coulter Z2 cell counter (Beckman Coulter, USA), BioRad TC20 cell counter (BioRad, USA), and a hemocytometer (Hausser Scientific, USA) were used to measure the number and concentration of cells and polystyrene beads and for cross validations. Human mesenchymal stem cells (MSCs) were purchased from Lonza (Lonza, Swiss). The log number used in this study was 0000471980 (derived from a 20-year-old male). MSCs were maintained in humidified incubators at 37° C. with 5% CO₂, and cultured with MSCs basal medium (Lonza) supplanted with 5% FBS. MSCs at passage 6 were cultured on 12-well plates with proper densities to avoid over confluency over a 6-day period. The initial number of cells for each condition is shown in Table 1 and Table 2. For hydrogen peroxide (H₂O₂) treatment, 30% H₂O₂ solution (Sigma, USA) were diluted with MSCs basal medium into desired concentrations. Media containing 100 μM and 200 μM H₂O₂ as well as the basal medium control were used to incubate MSCs at 37° C. for 2 h. After that, the MSCs were washed with 1×PBS solution 3 times and cultured in the fresh media for another 3 days before analysis. For X-ray treatment, MSCs were placed on a rotating table and exposed to 1 Gy, 4 Gy or sham (0 Gy), using a RAD320 320 kVp X-ray machine (Precision X-ray Inc., North Branford, Conn.), operated at 300 kV, 10 mA (dose rate of 1.3 Gy/min). Cells were cultured for another 3 days and 6 days, respectively, before analysis.

TABLE 1
Initial number of MSCs on a well for hydrogen peroxide treatment

| | 0 μM | 100 μM | 200 μM |
|---|---|---|---|
| 3 | 1 × 10⁴ | 1.5 × 10⁴ | 2 × 10⁴ |
| Days | cells/well | cells/well | cells/well |

TABLE 2
Initial number of MSCs on a well for X-ray irradiation

| | 0 Gy | 1 Gy | 4 Gy |
|---|---|---|---|
| 3 | 1 × 10⁴ | 2 × 10⁴ | 4 × 10⁴ |
| Days | cells/well | cells/well | cells/well |
| 6 | 5 × 10³ | 1 × 10⁴ | 2 × 10⁴ |
| Days | cells/well | cells/well | cells/well |

For mouse bone marrow samples, 10 weeks old, male wild-type mice (stain C57BL/6) were exposed to the total body X-ray irradiation at 0 Gy (sham), 1 Gy, 4 Gy, and 6.5 Gy, with 4 mice at each dose, respectively. The bone marrow samples were collected 10 days after the X-ray treatment and diluted with 1×PBS buffer to a total volume of ˜1.5 mL per mouse. For cell separation study, MSCs were dissociated with trypsin (Lonza, Swiss), fixed, and stored in 1×PBS buffer. Before being processed with a chip, the whole blood sample spiked with MSCs was filtered with a 40 μm cell strainer (Falcon, Corning, USA) to remove contaminants and clotting. To identify senescent MSCs, a Senescence Detection Kit (BioVision, Calif.) was used to stain senescent cells into blue color. To stain suspended MSCs, cells were incubated with the staining solution on chip or inside a tube at 37° C. overnight before study. For WBC study, 10 mL human whole blood was added to 200 mL 1×RBC lysis buffer (BioLegend, San Diego, Calif.) and incubated at room temperature for 15 min, followed by a centrifugation at 350×g for 5 min to enrich WBCs. The isolated WBCs were resuspended with 10 mL 1×PBS buffer and used to characterize the chips. The concentration of the input WBCs and recovered WBCs was measured with a Bio-Rad cell counter. To differentiate WBCs from the RBCs background, the nucleus of WBC was stained with Hoechst 33342 (Thermo Fisher Scientific, USA) and observed under a fluorescence microscope (350/461, DAPI). PE-CF594 mouse anti-human CD-45 antibodies (BD Horizon, USA) were used to label WBCs for fluorescence imaging.

Operation of chips. (1) A sample chip for analysis of senescent cells in whole blood. The chip with a 4 μm 3D filter array (or obstacle array) and a cell trapping array was used to isolate MSCs from whole blood, capture MSCs on chip, and conduct single cell analysis in situ after capture. 2 mL of fresh undiluted human whole blood spiked with ˜500 fixed senescent MSCs induced by either H₂O₂- or X-ray was injected into the chip at a flow rate of 3 mL/h. 1×PBS buffer with 0.05% BSA was injected from another inlet at a flow rate of 9 mL/h. After MSCs were captured on the cell trapping array, the flows of cell sample and buffer were stopped, and followed by a gentle injection of staining solution to fill the whole channel and tubing. The inlet tubing was kept during incubation to generate a balance pressure and prevent backflow of the trapped cells. During the separation and staining processes, air bubbles should be avoided inside the channel. After incubation, color images of the captured MSCs were recorded with the microscope for analysis. (2) A sample chip for removal of senescent cells from whole blood. A chip with a 13 μm 3D filter array (or obstacle array) was used to remove senescent MSCs from blood. Before spiked into human whole blood, the fixed senescent MSCs induced by either H₂O₂ or X-ray were stained overnight with the Senescence Detection Kit in a centrifuge tube. The percentage of the senescent MSCs was manually counted under the microscope by dropping 10 μL of the stained MSC sample on a glass slides. Then 10,000 stained MSCs were spiked into 3 mL undiluted human whole blood and run through the device at a flow rate of 3 mL/h. For the high-throughput separation device, the flow rate was increased to 300 mL/h. The removed senescent MSCs were collected in a tube from the outlet to measure the cell numbers and the percentage of senescent MSCs. The removed senescent MSCs were collected in a tube from the outlet to measure the cell concentration and the percentage of senescent MSCs. Therefore, the number of input and output senescent cells could be calculated. The removal rate was then determined by the ratio of output senescent MSCs over to input senescent MSCs.

Data analysis for chips. (1) Quantification of senescent cells on cell culture plates. To quantify the senescent MSCs on cell culture plates, the MSCs were fixed and then stained with Senescence Detection Kit and Hoechst 33342 (Thermo Fisher Scientific, USA). For each sample, five regions were randomly picked and recorded as a color image (RGB mode) and a fluorescent image (350/461, DAPI) using CCD camera on microscope with a 10× objective. The number of total MSCs and senescent MSCs were manually counted from the fluorescent images (DAPI) and color images (blue stain), respectively. Therefore, the percentage of senescent MSCs in each sample was determined by the ratio of senescent MSC number to total MSC number. (2)_Quantification of senescent cells on chips. After staining the MSCs on chip overnight, the color images of MSCs were recorded with a CCD camera in RGB mode. Microscope lamp intensity was consistent at 5 V. The images were then imported into ImageJ software to isolate their red channels, which were used to identify senescent MSCs. The grayscale of the dark region for each cell was measured with ImageJ, which define the senescent MSCs with a value smaller than 40.

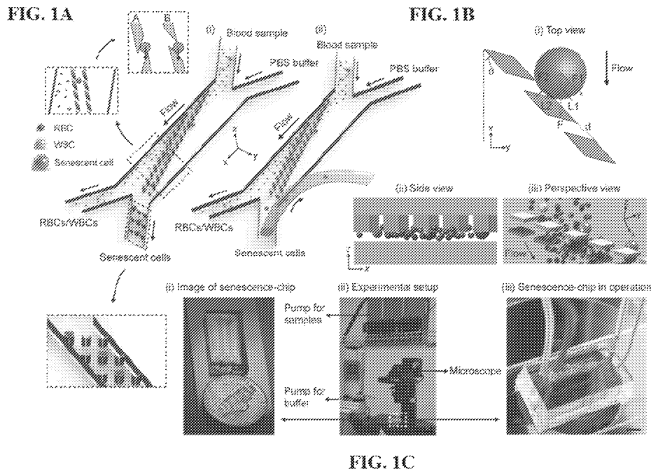

Mechanism of chips. FIGS. 1A-1C illustrate sample chips which monolithically integrate two rows of a tilted 3D filter array of obstacles for size-based cell separation with all necessary inlets and outlets for samples and buffers. Two types of chips were made for different purposes. For analysis of senescent cells in small volumes of whole blood or bone marrow, the chip contains a 3D-filter array (or obstacle array) to isolate MSCs, followed with a cell trap array to capture MSCs after separation for enumeration and single cell analysis of senescent cells (FIG. 1A(i)). For rapid removal of senescent cells from whole blood, the chip may not contain cell traps but a fluid outlet may be connected directly to a tubing to remove senescent cells from whole blood (FIG. 1A(ii)). The other end of the tubing may be connected to a waste or a collection tube for further analyses.

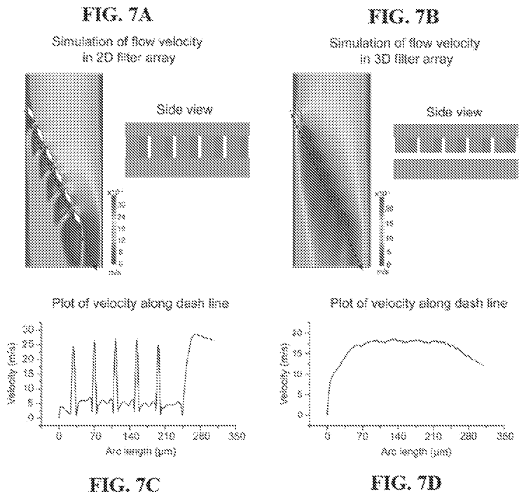

Modeling to optimize the configuration of the chips were shown in FIG. 1B and FIGS. 7A-7D. A 3D filter array (or obstacle array) is fabricated with PDMS micropillars inside the channel for cell separation on the x-y plane as well as in the z direction. On the x-y plane, two key parameters may be taken into consideration, which are the inclination angle (θ) of micropillars relative to the fluid flow, and the inter-pillar spacing (d) as shown in FIG. 1B(i). The pillar shape was also optimized to minimize clogging and maximize cell separation. Compared to the circular and triangle pillars, quadrangle pillars may have more uniformity in terms of flow pressure, deformability, and z-direction opening. Two types of quadrangle pillars were manufactured as shown in the zoom-in of FIG. 1A. When moving down the filter arrays (or obstacle arrays), the particle may be trapped by the sharp edge of the Type-A pillars. For Type-B pillars, the particle may contact a tilted surface on the pillars and may be easier to move on. Therefore, the Type-B pillars may have a better performance in particle separation. For the rigid particles with diameters smaller than the pillar spacing (d), they may directly pass through the filter (or obstacle). When a particle has a diameter larger than the pillar spacing, the hydrodynamic drag force (F) may be divided into two portions, parallel to the filter (F₁) and perpendicular to the filter (F₂). To ensure that the particles may roll down on the filter, the relation

F₁·L₁>F₂·L₂ (1)

may be established, in which L₁ and L₂ are the arms of forces F₁ and F₂. In Eq. (1), F₁ and F₂ may be expressed as F·cos θ and F·sin θ, while L₁ and L₂ may be expressed as (R²−¼d²)^{1/2} and ½d, in which R is the radius of the particle. Therefore, Eq. (1) could be expressed as

d<2R/(tan²θ+1)^{1/2} (2)

From Eq. 2, a smaller pillar spacing (d) and filter angle (θ) may help particles to roll down on the pillars as shown in FIG. 1A. In some cases, the angle of filter array (θ) may be 5°. As provided above and elsewhere herein, the pillar spacing (d) (also called “filter size” or “obstacle size”) may be varied based on the size of particles to be isolated.

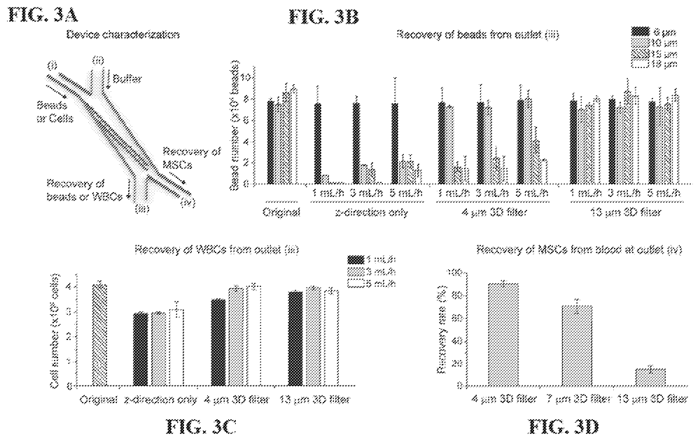

In the z direction, the PDMS pillars may not bond to the glass substrate because of their small top-surface area. Therefore, depending on the operational flow rate in the channel, an opening with varying size may be created between the pillars and the glass substrate, which may work as a shutter and allow for smaller particles (e.g., cells) to pass through (FIG. 1B). For example, during the separation of MSCs from whole blood, RBCs and WBCs can easily pass through the filter from both the z-direction and x-y plane, while the MSCs with a larger size may not cross the filter but instead roll down. As shown in FIGS. 7A-7D, as compared to the 2D filter array (or obstacle array) (FIGS. 7A and 7C), 3D filter array (FIGS. 7B and 7D) may generate much more uniform flow velocity across the channel. Therefore, the 3D filter array may better reduce the system backpressure, reduce clogging of the filter, and improve the throughput.

FIG. 1C shows the experimental setup for the operation of a sample chip. Two syringe pumps are used to deliver the 1×PBS buffer and blood samples into two inlets, respectively (FIG. 1C(ii)). A sheath flow of 1×PBS buffer may ensure the blood sample flow into the left outlet. As the cells in the blood sample flow down to the main channel, smaller cells such as RBCs and WBCs may pass the 3D filter array without changing their flow path. As a result, the smaller cells may exit to the left outlet. However, larger cells such as MSCs may be filtered out by the filters and roll down following the pillars to the right outlet (FIG. 1C(iii)).

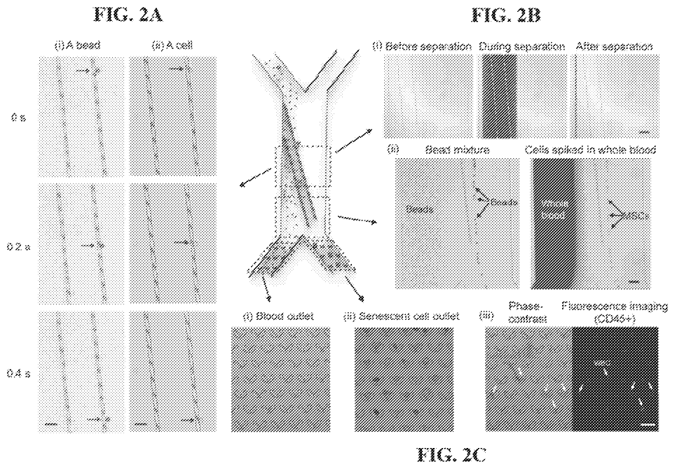

Performance of chips. The performance of sample chips is tested and testing results are shown in FIGS. 2A-2C. On a 4 μm 3D filter array (or obstacle array) (d=4 μm), time-lapse images clearly demonstrated that a 15 μm bead and a MSC rolled down on the filter array (FIG. 2A). In addition, when undiluted whole blood sample passed through the 3D filter array, no clogging was observed (FIG. 2B(i)). The separation ability of the chip with mixtures of polystyrene microbeads and MSCs spiked into undiluted fresh human whole blood is established (FIG. 2B(ii)). When 10 μm and 18 μm beads flowed down the channel, only the 18 μm particles may be filtered out by the filter while the smaller beads crossed the filter, as shown in the stacking image of FIG. 2B(ii) (left). After filtration, the 18 μm particles moving along the filter array were collected downstream from the right outlet. Similarly, the 4 μm 3D filter may isolate MSCs from blood cells, as shown in the stacking image of FIG. 2B(ii) (right). Although basal and senescent human MSCs may be heterogeneous in size, the microfluidic chip may still isolate most of them from blood cells due to their larger average sizes than RBCs and WBCs. After separation, MSCs were captured by the cell trap array located at the device outlet (FIG. 2C). On-chip SA-β-gal staining showed good separation of senescent MSCs (right outlet) and almost no background MSCs in blood cells (left outlet) (FIG. 2C(i)-(ii)). After labeled with anti-CD45 antibodies, background WBCs were able to be identified and excluded by comparing the phase-contrast image to fluorescence image (FIG. 2C(iii)).

Validation of chips. To further validate the separation ability of the chip and its dependence on 3D filter sizes (or obstacle sizes) and flow rates, device characterization using bead mixtures, isolated WBCs, and basal MSCs spiked in whole blood was performed (FIGS. 3A-3D). A mixture of beads and cells was received from the inlet (i) and the beads and cells which passed through the filter (or obstacle) from both the outlet (iii) and (iv) were recovered (FIG. 3A). The numbers of recovered beads and cells were measured. The bead mixture contained roughly an equal mix of 4 different sizes (6 μm, 10 μm, 15 μm, and 18 μm), and the total numbers of beads for each size was around 8.0-9.0×10⁴. As shown in FIG. 3B, most of the beads larger than 10 μm were removed by the z-direction only filter array (dam-like, with no openings in the x and y directions), while only 6 μm beads may pass the filter. As the flow rate increased from 1 mL/h to 5 mL/h, the number of beads larger than 10 μm also slightly increased, but was still lower than 25% of the original concentration. The results indicate that in 3D filter array, the spacing along the z-direction was smaller than 10 μm, but it may increase as the flow rate increased. With the 4 μm 3D filter array, more than 90% of the 6 μm and 10 μm beads may pass through the filter to outlet (iii), while larger particles were removed, suggesting the effective spacing along the z-direction was increased in the presence of openings in the x and y directions. As the pillar spacing increased to 13 μm, all size of beads from 6 μm to 18 μm may be recovered from outlet (iii), independent of the flow rates. Majority of the WBCs have a size between 8-12 μm. To test the ability of the chip to recover WBCs while removing senescent MSCs from whole blood, the RBC-lysed blood sample were used. The original (input) cell number of WBCs was around 4×10⁶. As shown in FIG. 3C, the z-direction only filter array allowed ˜75% of the WBCs to pass, while for the 4 μm and 13 μm 3D filter arrays, almost all of the WBCs may pass through and be recovered from outlet (iii). No WBCs were observed in cell traps at outlet (iv), confirmed by negative immunostaining with CD45. Presumably, the smaller WBCs were filtered through to the outlet (iii) or passed through the gap between the cell traps (˜10 μm) at outlet (iv), while the giant WBCs were pre-filtered by 40 μm cell strainer prior to on-chip separation.

Basal MSCs were spiked in whole blood and the original input number of MSCs was approximately 1×10⁴. The number of recovered MSCs was measured at outlet (iv) and the recovery rate was calculated, which is defined as the ratio between the recovered MSC number to the input MSC number. As shown in FIG. 3D, three types of 3D filter arrays (or obstacle arrays) with different filter sizes (pillar spacing of 4 μm, 7 μm, and 13 μm) were tested at a flow rate of 3 mL/h. The recovery rate of basal MSCs at outlet (iv) dropped from ˜90% to ˜20% as the 3D filter size increased from 4 μm to 13 μm.

Based on the results, 4 μm 3D filter arrays were used for analysis of senescent cells. Such filter size may isolate most of the MSCs from whole blood containing most of RBCs and WBCs, which may allow for quantification of the numbers and percentage of senescent MSCs among total MSCs. For removal of senescent cells from whole blood, it may be desirable to maximize the recovery of basal MSCs from outlet (iii) while only selecting senescent MSCs to outlet (iv), therefore 13 μm filter size may be utilized in this application.

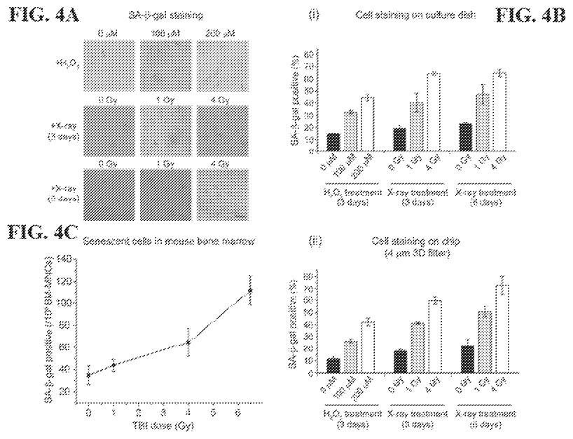

The microfluidic devices of the present disclosure may be used for analysis of senescent cells in human whole blood and mouse bone marrow samples (FIGS. 4A-4C). MSCs have been demonstrated to undergo in vitro cellular senescence by treatment of hydrogen peroxide and irradiation of X-ray. MSCs were treated with different doses of hydrogen peroxide (H₂O₂, 0, 100, 200 μM) and X-ray (0, 1, 4 Gy), and analyzed 3 days and 6 days after the treatments. Both dose-dependent and day-dependent increases of the percentage of SA-β-gal positive (stained blue) MSCs were observed on 12-well cell plates (FIG. 4A and FIG. 4B(i)). The H₂O₂- and X-ray-induced senescent MSCs (˜500 cells) were spiked in undiluted whole blood (˜2 mL) and underwent separation on the chip with a 4 μm 3D filter array at 3 mL/h, captured and stained on the single cell traps at outlet. Dose- and day-dependent increases of the percentage of SA-β-gal positive MSCs on the cell trap were observed, matching those determined by direct cell staining on culture dish (FIG. 4B(ii)). Importantly, the quantitation by the chips was achieved by starting with small numbers of MSCs and in the presence of undiluted human whole blood.

In some examples, the microfluidic devices (such as a microfluidic chip) may be used for separating or isolating senescent cells from mouse bone marrow. Four groups of mice were exposed to different doses of X-ray (0, 1, 4, 6.5 Gy). 10 days after TBI, the bone marrow was obtained and diluted into 1.5 mL with 1×PBS. About 1×10⁶ bone marrow mononuclear cells (BM-MNCs) from each sample was aliquoted and diluted into 2 mL for cell separation directly on the chips. As shown in FIG. 4C, the number of senescent cells isolated by the chips increased from the average 34 to average 112 as IR dose increased. Thus, the chips may be able to isolate and enumerate senescent cells from small volumes of various biofluids.

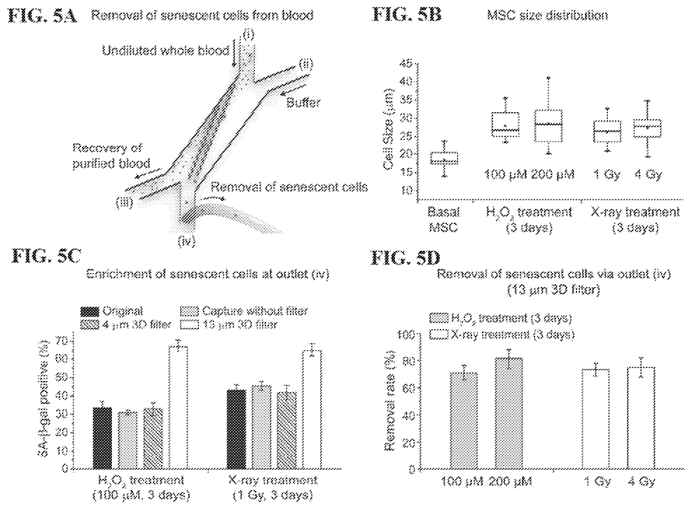

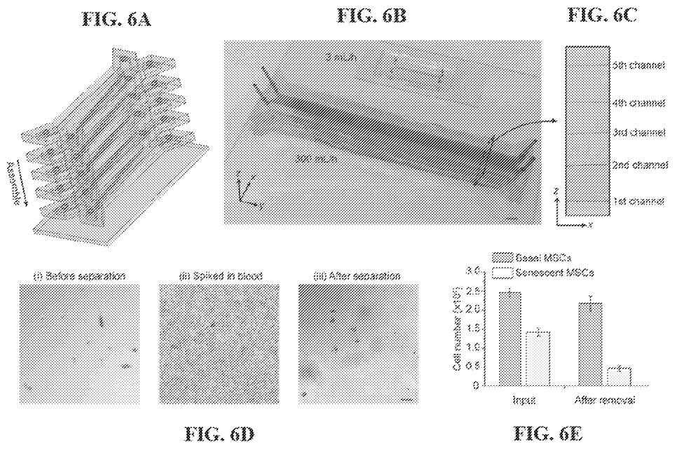

The microfluidic devices of the present disclosure may be used for removal of senescent cells from whole blood samples for potential therapeutic targeting of cellular senescence (FIGS. 5A-5D and FIGS. 6A-6E). The overall strategy was to maximize recovery of the major blood components including plasma, RBCs, WBCs, and healthy cells (in this case basal MSCs) from the outlet (iii), while removing most of the senescent MSCs through the outlet (iv) (FIG. 5A). To choose the filter size of 3D filter array for this application, it was determined that the average cell size of basal MSCs to be 18 μm and that of senescent MSCs to be above 25 μm (FIG. 5B). The enrichment efficiency by separating senescent MSCs from basal MSCs and captured them at outlet (iv) using 4 13 μm and 13 μm 3D filter arrays were shown in FIG. 5C. A control experiment was carried out by directly flowing the MSCs through the cell trap array to capture cells without a 3D filter. A significantly higher percentage (˜65%) of SA-β-gal positive MSCs were found at the outlet (iv) for 13 μm 3D filter array than the others. The result was consistent with those shown in FIG. 3D, where more than 80% of the basal MSCs were able to pass through the 13 μm 3D filter and recovered from outlet (iii). Therefore, the chip with a 13 μm 3D filter array was used for selective removal of senescent MSCs from basal MSCs and blood components. At a flow rate of 3 mL/h on this single-unit small-size chip, over 70% removal of pre-stained and SA-β-gal positive MSCs was achieved for both hydrogen peroxide and X-ray-induced senescent MSCs (FIG. 5D).

The microfluidic devices of the present disclosure may also be used to process samples with a higher throughput. As shown in FIGS. 6A-6C, the chip dimension may be scaled up by 10 folds. For example, the channel width may be increased from ˜10³ μm to ˜10⁴ μm. To prevent the deformation and collapse of the wide PDMS channel under high flow pressure, the chip was fabricated with posts uniformly distributed inside the channel. Five devices were stacked along the vertical direction for parallel processing to further improve the throughput. The multiplexed chips shared the same inlets and outlets to keep the operation simple. Given the dominant hydrodynamic resistance by the filter array over inlets and outlets, uniform flow rates were applied on each of five chips. With this multiplexed system, the throughput was increased by two orders of magnitude from 3 mL/h to 300 mL/h. To characterize the performance of the ultrahigh-throughput chip, 4×10⁴ pre-stained MSCs containing both senescent and non-senescent cells were spiked into 30 mL undiluted human whole blood for separation. As shown in FIGS. 6D and 6E, after separation, more than 70% of the senescent MSCs were removed while less than 15% of the basal MSCs were removed, demonstrating the similar performance from the high-throughput multi-unit chip as the single-unit small-size chip.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-50. (canceled)

51. A microfluidic device, comprising:
a fluidic channel; and
an array of obstacles disposed in said fluidic channel, and oriented at an angle greater than 0° relative to a direction of a fluid flow in said fluidic channel,
wherein an obstacle of said array of obstacles is oriented at an angle of less than 90° with respect to a surface of said obstacles, and wherein said array of obstacles is configured to separate one or more target analytes from a fluid flowing through said fluidic channel.

52. The microfluidic device of claim 51, wherein said array of obstacles is oriented at an angle between about 5° and 30° relative to said direction of said fluidic flow.

53. The microfluidic device of claim 51, wherein a distance between said array of obstacles and a side wall of said fluidic channel increases along said direction of said fluid flow.

54. The microfluidic device of claim 51, wherein individual obstacles of said array of obstacles have a quadrilateral cross-section.

55. The microfluidic device of claim 54, wherein said quadrilateral cross-section is a parallelogram cross-section.

56. The microfluidic device of claim 51, wherein each obstacle of said array of obstacles is oriented at said angle of less than 90° with respect to said surface of said obstacles.

57. The microfluidic device of claim 51, wherein an average spacing size between obstacles of said array is between about 100 nanometers and 100 micrometers (μm).

58. The microfluidic device of claim 51, wherein said array of obstacles has a height less than or equal to a height of said fluidic channel

59. The microfluidic device of claim 51, wherein said array of obstacles is configured to direct said one or more target analytes to flow at a direction different from said direction of said fluid flow.

60. The microfluidic device of claim 51, wherein said array of obstacles is configured to separate said one or more target analytes from said fluid based at least partially on a size of said one or more target analytes.

61. The microfluidic device of claim 51, wherein said array of obstacles comprises three-dimensional (3D) microstructures.

62. The microfluidic device of claim 51, wherein at least a subset of said array of obstacles is configured to deform when a flow rate of said fluid is greater than a threshold value.

63. The microfluidic device of claim 51, wherein obstacles of said array of obstacles are non-porous.

64. The microfluidic device of claim 51, further comprising an additional fluidic channel in fluidic communication with said fluidic channel.

65. The microfluidic device of claim 64, further comprising an additional array of obstacles disposed within said additional fluidic channel, which additional array of obstacles is configured to capture and retain said one or more target analytes.

66. The microfluidic device of claim 65, wherein an individual obstacle of said additional array of obstacles has an opening, which opening has a dimension greater than or equal to a size of said one or more target analytes.

67. The microfluidic device of claim 51, comprising a plurality of microfluidic channels, each comprising a different array of obstacles, configured to separate a given type of target analyte from a plurality of types of target analytes within said fluid, wherein said one or more target analytes are of said given type.

68. The microfluidic device of claim 51, comprising an outlet in fluidic communication with an inlet of one or more additional microfluidic devices.

69. The microfluidic device of claim 51, wherein said microfluidic device and said one or more additional microfluidic devices are connected in parallel, in series or in a combined configuration of in series and in parallel.

70. A microfluidic device, comprising:
a fluidic channel; and
an array of obstacles disposed in said fluidic channel;
wherein said array of obstacles is configured to separate one or more senescent cells from a fluid having a volume less than or equal to about 1 milliliter (mL) at an efficiency greater than about 70% upon flow of said fluid through said fluidic channel.

71. A method, comprising:
(a) directing a fluid comprising one or more target analytes into a microfluidic device, said microfluidic device comprising:
a fluidic channel; and
an array of obstacles disposed in said fluidic channel, wherein said array of obstacles is oriented at an angle greater than 0° relative to a direction of a fluid flow in said fluidic channel;
(b) directing said fluid to flow through said fluidic channel; and
(c) separating at least a portion of said one or more target analytes from said fluid using said array of obstacles upon flow of said fluid through said fluidic channel.
